(19) United States
(12) Patent Application Publication
(10) Pub. No.: US 2023/0049525 A1
(43) Pub. Date: Feb. 16, 2023

(54) METHODS OF IDENTIFYING CELL-TYPE-SPECIFIC GENE EXPRESSION LEVELS BY DECONVOLVING BULK GENE EXPRESSION

(71) Applicant: The United states of American, as Representative by the Secretary, Dept.of Health and Human Services, Bethesda, MD (US)

(72) Inventors: Kun Wang, Bethesda, MD (US); Sushant Patkar, Bethesda, MD (US); Joo Sang Lee, Bethesda, MD (US); Eytan Ruppin, Bethesda, MD (US); Alejandro Schaffer, Bethesda, MD (US)

(21) Appl. No.: 17/780,356

(22) PCT Filed: Nov. 25, 2020
(86) PCT No.: PCT/US2020/062238

§ 371 Date: May 26, 2022

Related U.S. Application Data
(60) Provisional application No. 62/940,755, filed on Nov. 26, 2019.

(51) Int. Cl. G16B 25/10; G16B 35/20; G16B 5/20; G16H 50/20

(52) U.S. Cl. CPC G16B 25/10; G16B 35/20; G16B 5/20; G16H 50/20

(57) ABSTRACT

Provided herein are methods of identifying gene expression levels in specific cell types based on bulk gene expression levels measured in tissue samples comprising a plurality of cell types.

### STATEMENT REGARDING FEDERALLY SPONSORED RESEARCH OR DEVELOPMENT

This invention was made with Government support under project number ZIA BC 011803 by the National Institutes of Health, National Cancer Institute. The United States Government has certain rights in the invention.

### FIELD OF THE INVENTION

Provided herein are methods of identifying gene expression levels in specific cell types based on bulk gene expression levels measured in tissue samples comprising a plurality of cell types.

### BACKGROUND OF THE INVENTION

The importance of the tumor microenvironment (TME) in cancer has been recognized since the late 1800s. The recent success of immune checkpoint blockade has further sparked interest in studying TME interactions that shape clinical outcomes following immunotherapy, aiming to find biomarkers of treatment response and new treatment opportunities [2]. One key step in studying these interactions is the characterization of the molecular profiles of different cell types in a given patient's tumor sample. Fluorescence-activated cell sorting (FACS) and single cell RNA sequencing have emerged as effective tools to address this challenge [3]. However, due to the cost and scarcity of fresh tumor biopsies, the application of these approaches remains limited. Given that bulk tumor gene expression from preserved biopsies is far more abundant, computational methods that can effectively extract cell-type-specific expression from such data, termed deconvolution algorithms, could be very helpful. If successful, such deconvolution methods can markedly advance our knowledge of the TME across many tumor types and different contexts, and beyond that, they may be readily applied to interrogate other large bulk expression datasets.

Several previous studies have developed a variety of expression deconvolution algorithms. DeMixT [4] was designed to estimate individual-specific expression for three cell components with provided prior reference samples of two cell components. ISOpure [5] has aimed to derive individual-specific cancer cell expression with the assumption that the observed bulk gene expression profile is a mixture of predefined stromal and immune cell expression profiles that are shared across all the individuals. Building on this work, Fox et al extended ISOpure to predict individual-specific non-tumor cell expression by subtracting cancer cell profiles from the bulk mixtures in a two-cell type model [6]. More recently, A. M. Newman et al. (“Determining cell type abundance and expression from bulk tissues with digital cytometry,” Nat. Biotechnol., vol. 37, no. 7, pp. 773-782, 2019, the contents of which are incorporated herein) developed CIBERSORTx, the first approach that aims to predict the sample-specific gene expression of all cell types by employing a set of novel deconvolution heuristics. As a proof of concept, Newman et al. showed that CIBERSORTx can accurately reconstruct the cell-type-specific expression of genes in each input sample under certain modelling assumptions. This groundbreaking work has, however, some notable limitations: (1) The number of genes whose cell-type-specific expression can be reconstructed in each sample is relatively small, especially for low-abundance cell types, and (2) their approach does not provide confidence estimations of the predictions made, while such estimations are important in most deconvolution applications in the absence of ground truth data.

Accordingly, there is a need in the art for improved methods for identifying cell-type-specific gene expression levels from bulk gene expression data in a sample containing multiple cell types.

### SUMMARY OF THE INVENTION

Provided herein is a method of identifying cell-type specific gene expression levels for specific cells in a plurality of tissue samples. The tissue samples may be obtained from a set of tumor samples. The cell types may comprise tumor, immune, and stromal cell types.

The method may comprise (a) receiving a collection of bulk gene expression level measurements and cell fractions for each cell type in each of the tissue samples in a given collection of samples, obtained from a set of tissue samples of a plurality of cell types; (b) performing high resolution deconvolution on the bulk gene expression measurements and cell fractions for each sample to generate a first output comprising predicted cell-type-specific gene expression levels in each cell type; (c) ranking the first output with a confidence ranking system to generate a first output ranking of genes with predicted cell-type-specific gene expression levels of low or high confidence; (d) performing hierarchical deconvolution the genes of low confidence from step (c) to generate a second output, such that for each gene of low confidence from step (c), the expression of the gene in a specific cell type is re-estimated by removing expression of genes of high confidence in all other cell types from the bulk gene expression measurements, thereby generating a second output comprising genes with predicted cell-type-specific gene expression levels of low or high confidence; (e) ranking the second output with the confidence ranking system to generate a second output ranking of genes with predicted cell-type-specific gene expression levels of low or high confidence; (f) performing imputation based deconvolution on the genes of low confidence from step (e) to generate a third output, such that for each gene of low confidence from step (e) for which gene expression levels in a particular cell type are highly correlated with the bulk gene expression levels of more than two genes of high confidence, a Lasso regression-based prediction model based on the bulk gene expression levels is applied, thereby imputing the expression of the low confidence genes based on the expression of high confidence genes from the first output ranking; (g) ranking the third output with the unsupervised ranking system to generate a third output ranking comprising genes with predicted cell-type-specific gene expression levels of low or high confidence; and, (h) generating a final confidence score for each pair of predicted gene expression levels and specific cell type, based on the predicted cell-type-specific gene expression levels of each gene of low or high confidence as determined in step (g) in comparison to the predicted cell-type-specific gene expression level of each gene of high confidence as determined in step (g). The method may thereby generate deconvolved cell-type-specific gene expression data identifying cell-type-specific gene expression levels for each cell type in each of the individual tissue samples.

One or more of the first output ranking, the second ranking, and third output ranking for each gene may be a 1 or 0. A ranking of 1 may indicate the output is high confidence and the ranking of 0 may indicate the output is low confidence. The high resolution deconvolution may comprise determining the cell types in which the genes are weakly expressed; recursively splitting the samples into finite sub-groups using p-freedom based splitting; and, performing ensemble sliding window deconvolution.

A final confidence score for each gene-specific cell type may be determined by: (i) for each gene, averaging the pair-wise correlations between the predicted gene expression levels of the gene' in each specific cell type and the predicted expression level of genes of high confidence as determined in step (g) described above; (ii) randomly shuffling the predicted cell-type-specific gene expression levels across the samples to generate a background and repeating step (i) to estimate a background distribution of scores for each gene; (iii) for each gene and each cell type, determining an empirical p-value pv based on the background distribution of scores; and, (iv) for each gene-cell type pair, subtracting pv from 1 to generate the final confidence score for the predicted gene expression levels of each gene in each specific cell type.

The confidence ranking system may comprise ranking a prediction based on each informative feature/measurement collected during or after each deconvolution step independently. The confidence ranking system may also use the correlation between cell fraction and bulk expression, Spearman correlation between predicted cell-type-specific expression and bulk gene expression across samples, variations among groups from recursive splitting deconvolution, consistency between sliding windows and recursive splitting deconvolution for confidence ranking of the first output. The confidence ranking system may further use mean correlations with highly predictable genes from the first output. The confidence ranking system may use mean correlations with genes of high confidence from both the first output and the second output.

The cell fractions of each cell type may be determined by receiving bulk gene expression measurements and cell-type-signature profiles; and, performing support vector machine (SVM) regression on the bulk gene expression measurements and cell-type-signature profiles; thereby determining the cell fractions of each cell type. Determining the cell fractions of each cell type may comprise performing batch correction on the bulk gene expression measurements and cell-type-signature profiles.

Also provided herein is a method of identifying ligand-receptor interactions between a first cell type and a second cell type from a tissue sample. The method may comprise: (a) querying ligand-receptor interactions between the first cell type and second cell type from a database comprising a catalog of ligand-receptor interactions among a plurality of cell types and an expected distribution of ligand and receptors on each of the plurality of cell types, thereby generating a first list of potential ligand-receptor interactions between the first cell type and the second cell type; (b) recursively adding to the first list ligands and receptors that are expected to be found on generic cell types related to the first and second cell types by function or lineage, or a combination thereof, thereby generating a second list of potential ligand-receptor interactions between the first cell type and the second cell type; (c) receiving deconvolved cell-type-specific gene expression data according to any of the preceding claims; (d) determining the likelihood of each potential ligand-receptor interaction between the first cell type and second cell type by assigning a binary score to the interaction, wherein based on the deconvolved cell-type-specific gene expression data, the binary score is 1 if the ligand is overexpressed in the first cell type and the receptor is overexpressed in the second cell type, and the binary score is 0 otherwise; and, (e) inferring the activity of queried ligand-receptor interactions using the cell-type-specific gene expression levels. The method may thereby identify ligand-receptor interactions between cell types.

The tissue sample may be a tumor sample. The ligand-receptor interactions may comprise at least one of cytokine/chemokine-cytokine/chemokine receptor interactions, ligand-receptor interactions involved in cell adhesion/leukocyte trans-endothelial migration, ligand-receptor interactions involving the TNF receptor superfamily, and ligand receptor interactions involved in regulation of NK and T cell cytotoxicity. The ligand may be overexpressed in the first cell type and the receptor is overexpressed in the second type in comparison to, based on the deconvolved cell-type gene expression data, the median deconvolved expression of the ligand in the first cell type and the median deconvolved expression of the receptor in the second cell type.

The method may further comprise determining if a ligand-receptor interaction is more likely to occur in a tissue sample with a specific phenotype as compared to a control group, by computing an enrichment score, wherein the enrichment score is expressed as an odds ratio of the interaction in the specific phenotype, wherein a score around 1 indicates a neutral trend, a score>1 indicates enrichment of the interaction in the specific phenotype, and a score close to 0 indicates enrichment in the control group.

Further provided herein is at least one non-transitory computer readable medium storing instructions which when executed by at least one processor, cause the at least one processor to: (a) receive a collection of bulk gene expression level measurements and cell fractions for each cell type in a set of tissue samples of a plurality of cell types; (b) perform high resolution deconvolution on the bulk gene expression measurements and cell fractions for each sample to generate a first output comprising predicted cell-type-specific gene expression levels in each cell type; (c) rank the first output with a confidence ranking system to generate a first output ranking of genes with predicted cell-type-specific gene expression levels of low or high confidence; (d) perform hierarchical deconvolution the genes of low confidence from step (c) to generate a second output, such that for each gene of low confidence from step (c), the expression of the gene in a specific cell type is re-estimated by removing expression of genes of high confidence in all other cell types from the bulk gene expression measurements, thereby generating a second output comprising genes with predicted cell-type-specific gene expression levels of low or high confidence; (e) rank the second output with the confidence ranking system to generate a second output ranking of genes with predicted cell-type-specific gene expression levels of low or high confidence; (f) perform imputation based deconvolution on the genes of low confidence from step (e) to generate a third output, such that for each gene of low confidence from step (e) for which gene expression levels in a particular cell type are highly correlated with the bulk gene expression levels of more than two genes of high confidence, a Lasso regression-based prediction model based on the bulk gene expression levels is applied, thereby imputing the expression of the low confidence genes based on the expression of high confidence genes from the first output ranking; (g) rank the third output with the unsupervised ranking system to generate a third output ranking comprising genes with predicted cell-type-specific gene expression levels of low or high confidence; and, (h) generate a final confidence score for each pair of predicted gene expression levels and specific cell type, based on the predicted cell-type-specific gene expression levels of each gene of low or high confidence as determined in step (g) in comparison to the predicted cell-type-specific gene expression level of each gene of high confidence as determined in step (g). The processor may thereby generate deconvolved cell-type-specific gene expression data identifying cell-type-specific gene expression levels for each cell type in the tissue samples. The at least one non-transitory computer readable medium may store further instructions which when executed by at least one processor, cause the at least one processor to perform one or more of the steps of any one of the methods described above.

### BRIEF DESCRIPTION OF THE DRAWINGS

FIG. 1A-B. Overview of methods described herein, including COnfident DEconvolution For All Cell Subsets (CODEFACS) and LIgand Receptor Interactions between Cell Subsets (LIRICS). FIG. 1A. CODEFACS takes bulk gene expression profiles and prior knowledge of the cellular composition of each sample and executes a greedy three step algorithm to infer the deconvolved gene expression in each sample. In module 1, a high-resolution deconvolution is performed. In module 2 (hierarchical deconvolution), bulk expression is modeled as a mixture of two components: a specific cell-type of interest and all the remaining cell types. The expression for the cell type of interest is predicted by removing the estimated expression in the second component (using high-resolution deconvolution from module 1) from the bulk mixture. In module 3—imputation-based deconvolution, the cell-type-specific expression of a specific gene is imputed based on the predicted cell-type-specific expression of other high-confidence genes that are co-expressed with that gene in the bulk. Each module is designed to overcome the shortcomings of its predecessor based on their respective modeling assumptions. The confidence ranking system is responsible for classifying all the predictions at the end of each module into high-confidence or low-confidence predictions. Genes classified into the low-confidence class at the end of one module (e.g. module 1) are passed to the next module (e.g. module 2) for refinement. Finally, after all the three modules are executed, the prediction confidence levels are re-evaluated. The final output of CODEFACS consists of a 3-dimensional matrix with cell-type-specific gene expression predictions for each sample, along with estimated confidence scores of predictions for each gene in each cell type.

FIG. 1B. LIRICS takes the output of CODEFACS and processes it in three steps. In step 1, for each possible permutation of cell type pairs, LIRICS queries a literature-curated repository for enumerating all plausible ligand-receptor interactions between specified cell types. In step 2 this prior knowledge is integrated with the output from CODEFACS to infer which of the plausible cell-cell interactions are likely to occur or be “active” in each individual sample. The result is a binary matrix with rows representing each plausible cell-cell interaction and columns representing each patient's tumor sample. Finally, in step 3, given any clinically relevant phenotype of interest (e.g. response to therapy, driver mutation status, etc.), one can perform a Fisher's enrichment analysis (shown at the bottom) to discover cell-grounded receptor-ligand interactions in the TME that are associated with the phenotype of interest.

FIG. 2. Given the bulk mixture matrix B (m genes×n samples) and cell type signature profile S (h genes×c cell types), SVM regression is used to predict the cell fraction matrix F (c cell types×n samples).

FIG. 3. Given initial estimates of cell fractions F (c cell types×n samples) and cell type signature profile S (m genes×c cell types), one can reconstruct the bulk expression matrix via the matrix multiplication S×F. Batch effects are then reduced between the given bulk subset B′ and reconstructed bulk S×F.

FIG. 4. Recursive splitting method. Given the estimated cell fractions F (c cell types×n samples) and sorted bulk expression of gene i, the adequacy of the sample size is checked for NNLS first: if not, it will exit; if yes, two-freedom splitting will be performed. Subsequently, it is checked whether the two-freedom splitting improves the bulk reconstruction. If yes, both the low expressed and high expressed groups will recursively enter another round of two-freedom splitting; if no, the two-freedom splitting based predictions will be ignored and the function exits.

FIG. 5A-C. Gene-gene correlations among cell types. FIGS. 5A-C are boxplots depicting the gene-gene expression correlation distributions among cell types in SKCM dataset 1, GBM dataset 1 and LUAD dataset respectively for 1000 randomly selected genes. In each of the three plots, corresponding random-permutation-based background controls are provided. The light shaded box represents the correlation derived from the original datasets as the foreground (fg), while the darker box represents that derived from the randomly permuted background control (bg). The y-axis denotes the Spearman correlation value and the x-axis denotes the cell type.

FIG. 6. Hierarchical deconvolution. Given the estimated cell fractions F (c cell types×n samples), bulk and confidence levels estimated from module 1, for each cell type k all the other cell types are merged as a pseudo component to construct a two-component model. Thereafter for each low-confidence gene i in cell type k, module 1 is run to predict the expression in the pseudo component and finally remove the estimated expression of the pseudo component from the bulk to estimate the expression of the low-confidence gene i in cell type k.

FIG. 7. Imputation-based deconvolution. Given the predicted cell-type-specific expression G (m genes×n samples×c cell types), bulk and confidence levels estimated from module 1, in each cell type k and for each low-confidence gene i, the correlation between gene i and each of other genes in bulk is computed. If the number of genes which are highly correlated with gene i is more than 2, a machine learning model is built up to predict the expression of gene i in cell type k based on the expression of other high-confidence genes which are correlated with gene i. After imputation, both the predicted expression matrix G and confidence matrix C will be updated.

FIGS. 8A-I. Evaluating the performance of CODEFACS. FIGS. 8A-C show bar plots depicting the number of high confidence predicted genes (Kendall correlation≥0.3) in each cell-type, estimated from bulk-generated samples of lung cancer (LUAD dataset; sample size=26), melanoma (SKCM dataset 1; sample size=28) and glioblastoma (GBM dataset 1; sample size=24) benchmark datasets, as estimated by CODEFACS (light bars) and CIBERSORTx (dark bars). FIGS. 8D-F show boxplots depicting prediction accuracy distributions of all genes across different cell types in the lung cancer (LUAD with sample size 26), melanoma (SKCM with sample size 28) and glioblastoma (GBM with sample size 24) benchmark datasets, using CODEFACS (light) and CIBERSORTx (dark). A two-sided Wilcoxon signed rank test was performed to compare the prediction accuracies of CODEFACS and that of CIBERSORTx for each cell type in each dataset. *** denotes p-values<2e⁻¹⁶. FIG. 8G shows Spearman correlations between prediction accuracies and confidence scores among cell types in the lung cancer benchmark dataset (LUAD dataset; sample size=26). The y-axis indicates the spearman correlation coefficient value, while the x-axis indicates the cell type. FIG. 8H shows AUCs obtained in classifying informative and uninformative predictions among cell types in lung cancer benchmark dataset (LUAD dataset; sample size=26). FIG. 8I shows bar plots depicting the Spearman correlations between mean deconvolved cell-type-specific expression in TCGA-LUAD and mean cell-type-specific expression derived from publicly available single cell datasets of LUAD. The y-axis indicates the spearman correlation coefficient value, while the x-axis indicates the cell type.

FIGS. 9A-D. FIG. 9A shows the landscape of tumor mutation burden and microsatellite instability across 18 different solid tumor types. This panel plots the distribution of non-synonymous tumor mutation burden on a logarithmic scale (Y-axis). All points above the horizontal line are typically regarded as hyper-mutated tumors (>10 mutations/Mb). All dark grey points represent tumors with a DNA mismatch repair deficiency detected via microsatellite instability (MSI). FIG. 9B depicts the fraction of all tumor samples per cancer type with microsatellite instability (Y-axis). Tumor types marked with a * represent those where MSI is prevalent. FIG. 9C-D show comparisons of overall survival of patients with tumors that are hypermutated vs not hypermutated. Left panel (FIG. 9C): Analyzes solid tumor types where hypermutated tumors frequently have MSI (marked with a * in panel FIG. 9B). Right panel (FIG. 9D): Analyzes solid tumor types where hypermutated tumors rarely have MSI. Statistical significance of differences in survival was calculated using the log-rank test.

FIG. 10A shows an interaction network consisting of the top 50 interactions most highly enriched in TME of tumors with DNA mismatch repair deficiency. Interactions highlighted in lighter grey represent co-stimulatory interactions/having an activating effect on the target cell. Interactions highlighted in dark grey represent checkpoint interactions/having an inhibitory effect on the target cell. Interactions highlighted in black represent pro-inflammatory/chemotaxis interactions involved in inflammatory response and immune cell trafficking to tumor sites. Eos: Eosinophils, CAF: Cancer associated fibroblasts.

FIG. 10B shows a volcano plot depicting on the x-axis the log 2 fold change in the frequency of occurrence of each cell-cell interaction in the TME of hypermutated tumors with a frequent underlying DNA mismatch repair deficiency vs hypermutated tumors with almost no underlying DNA mismatch repair deficiency. The y-axis indicates the −log 10 FDR adjusted p-value of the observed enrichment. Highlighted in darker grey in the scatter plot are the interactions shown in FIG. 10A.

FIG. 11A shows an overview of the analysis employed to identify mutation specific functional interactions (MSFI) ligand-receptor interactions cell type specific interactions that are predictive of immune checkpoint blockade therapy.

FIG. 11B shows a chord diagram of the MSFI network. Each individual interaction is represented by a link from the source cell type (ligand expressing cell type) to the target cell type (receptor expressing cell type) and the shade of the link represents the shade of the source cell type. For interactions that are activating/co-stimulatory, the sector in the corresponding target cell type is highlighted in a medium shade. For inhibitory/checkpoint interactions, the sector in the target cell type is in highlighted in a lighter shade. Interactions involved in chemotaxis are highlighted in black and those mediating a pro-inflammatory response are highlighted darkest. The thickness of each link is proportional to the fold change in frequency of occurrence of the interaction in patients with RECISTv1.1 Partial/Complete response.

FIG. 11C shows Kaplan-Meier plot depicting the progression free survival of all melanoma patients receiving immune checkpoint blockade (N=244). On the top, the patients are stratified into low-risk/high-risk groups based on the median value of MSFI score from LIRICS. Second from top, patients stratified into low/high risk groups based on median IMPRES score [34]. Third from top, patients stratified into low/high risk groups based on median TIDE score [35], Bottom, patients stratified into low/high risk groups based on median MPS score [36].

FIG. 11D shows Kaplan-Meier plots depicting the overall survival of all melanoma patients receiving immune checkpoint blockade (N=244). On the top, the patients are stratified into low-risk/high-risk groups based on the median value of cellular crosstalk score from LIRICS. Second from top, patients stratified into low/high risk groups based on median IMPRES score [34]. Third from top, patients stratified into low/high risk groups based on median TIDE score [35], Bottom, patients stratified into low/high risk groups based on median MPS score [36].

FIG. 11E shows area under the ROC curves in predicting Complete/Partial-response (based on RECIST v1.1) to immune checkpoint blockade therapy for the different scores. X-axis marks patients grouped by dataset source and treatment regimen. PD1 mono represents patients that received anti-PD1 monotherapy. PD1+CTLA4 represents patients that additionally received anti-CTLA4 besides anti-PD1.

FIGS. 12A-F. Performance comparison between two algorithm settings. FIGS. 12A-F show bar plots depicting the number of highly predictable genes (Kendall correlation≥0.3) using the two algorithmic approaches (ensemble of windows with p-splitting smoothing and single window with 2-splitting smoothing) in each cell type among six benchmark datasets with sufficient sample size (100). The dark bar represents the performance of ensemble of windows with p-splitting smoothing, while the white bar represents that of single window with 2-splitting smoothing.

FIGS. 13A-O. The number of highly predictable genes (Kendall correlation≥0.3) in each cell-type among the 15 benchmark datasets across the three algorithm steps. FIGS. 13A-O show bar plots depicting the number of highly predictable genes (Kendall correlation≥0.3) in each cell type among the 15 benchmark datasets respectively. The light shaded bar represents the performance of module 1, the white bar represents that of module 2 and the dark one represents that of module 3. The y-axis indicates the number of highly predictable genes and the x-axis indicates the cell types.

FIGS. 14A-O. Prediction accuracy distributions across three algorithms steps in each cell-type among the 15 benchmark datasets. FIGS. 14A-O show boxplots depicting prediction accuracy distributions of high-confidence genes (based on CODEFACS) among cell types respectively in the 15 benchmark datasets. The light shaded bar represents the performance of module 1, the white bar represents that of module 2 and the dark one represents that of module 3. The y-axis indicates the prediction accuracy and the x-axis indicates the cell types.

FIG. 15 is a block diagram illustrating an example of a computing device or computer system 600 which may be used in implementing the embodiments of the components of the methods disclosed herein.

### DETAILED DESCRIPTION

Described herein is a deconvolution tool, CODEFACS (COnfident DEconvolution For All Cell Subsets) that markedly advances the ability to successfully deconvolve bulk gene expression data. Building on its enhanced coverage and accuracy, it is the first to be applied to deconvolve and analyze cancer expression data in a sample-specific manner on a large scale. CODEFACS receives as input bulk gene expression profiles of tumor samples and prototypical molecular signatures of expected tumor, immunological and stromal cell types, which serve as seeds for estimating the abundance of each of the cell types in each sample. Given these inputs, CODEFACS estimates the different cell-type-specific gene expression profiles and their abundances in each sample. It is a greedy algorithm aimed at maximizing the number of genes in each cell type whose expression across the samples can be confidently predicted. CODEFACS robustly improves over a previously-described method referred to as CIBERSORTx, both in terms of gene coverage and individual gene expression estimation accuracy. The methods for CIBERSORTx are described in U.S. Patent Application Publication No. 2020/0176080, the contents of which are incorporated herein by reference.

In particular, the presently disclosed methods improve upon CIBERSORTx by including a high resolution deconvolution step that applies a recursive splitting method to reconstruct observed bulk gene expression data (also referred to as a p-freedom approach), and also applies ensemble sliding windows deconvolution. In addition, the methods disclosed herein use a confidence ranking system that classifies predictions into high- or low-confidence, thereby making it possible to further refine low-confidence predictions. The confidence ranking system enables evaluation of predictions, and provides a confidence score for each pair of genes and cell types. The confidence score correlates with prediction accuracy, and is useful for removing uninformative predictions. Such a confidence ranking system has not been previously implemented in known methods. The methods disclosed herein additionally include hierarchical deconvolution and imputation-based deconvolution, both of which improve and refine predictions of cell-type-specific gene expression levels as compared to previously known approaches.

Also described herein is a method called LIRICS (LIgand Receptor Interactions between Cell Subsets), a pipeline that integrates the output of CODEFACS with a database of prior immunological knowledge that has been curated to infer the active cell-cell interaction landscape in each sample. These data can then be analyzed in conjunction with any sample-associated clinical annotations (e.g., response to treatment) to infer the most important clinically relevant immune interactions between the cell types in a given cancer cohort.

CODEFACS has been applied to reconstruct the cell-type-specific transcriptomes of 7824 tumor samples from 21 cancer types in the Cancer Genome Atlas (TCGA). Analyzing these fully deconvolved TCGA expression datasets using LIRICS, the inventors have found a shared repertoire of intercellular interactions enriched in the TME of mismatch repair deficient tumors of different tissues of origin, which is associated with improved overall patient survival and high response rates to anti-PD1 treatment. Using machine learning techniques, the inventors have further identified intercellular TME interactions that are predictive of response to immune checkpoint blockade treatment in melanoma patients.

In summary, CODEFACS and LIRICS present a new way to analyze large publicly available bulk RNA-seq datasets to study cellular crosstalk in the TME of each patient, and to learn more about its association with different clinical measures. The potential scope of applications of both CODEFACS and LIRICS goes beyond studying the TME, as these tools can be applied to study any disease of interest given bulk gene expression data and relevant reference signatures of cell types involved.

### 1. Definitions

The terminology used herein is for the purpose of describing particular embodiments only and is not intended to be limiting. As used in the specification and the appended claims, the singular forms “a,” “an” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6,9, and 7.0 are explicitly contemplated.

### 1. CODEFACS Algorithm

Provided herein is a method of identifying cell-type-specific gene expression levels and cell type abundance in tissue samples. The method implements a greedy divide-and-conquer algorithm to reconstruct cell-type-specific transcriptomes from individual bulk mixtures in silico. The method may comprise providing one or more of the following: (1) bulk gene expression of a collection of samples (required); (2) cell fraction estimates of expected cell types in each sample, which may comprise one or of tumor, immune and stromal cell-types (implemented if cell type specific signature profile is not provided); and, (3) a cell-type specific signature profile (required if cell fractions are not provided). The gene expression data may be RNA sequencing (RNA-seq) data, and may be obtained from the Cancer Genome Atlas (TCGA). The method may generate one or more of the following outputs: (1) a prediction of the expression level of each gene in each sample in each cell-type in the mixture; and, (2) confidence scores for each gene-cell-type pair, which denotes the confidence level in the predicted expression of a gene in a cell type across samples. The confidence score may be 0-1, where ˜1 indicates high confidence, and ˜0 indicates low confidence.

In one example, the following computational, full deconvolution problem is solved:

minimizeΣᵢ₌₁^{m}∥(B_{i,}.−diag(Gᵢ,.,.×F^{T}))∥ (1)
subject to Σₖ₋₁^{c}fⱼₖ=1 for all j
   gᵢⱼₖ≥0 for all i, j, k
   fⱼₖ≥0 for all j, k

where B represents the given bulk RNA-seq expression matrix (m genes×ⁿ samples), in which each entry bᵢⱼ is the observed bulk expression for i^{th} gene and j^{th} sample; G is a three-dimensional deconvolved expression matrix (m genes×n samples×c cell types), in which gᵢⱼₖ denotes the unknown expression for gene i in the j^{th} sample and k^{th} cell type; F is the cell fraction matrix (n samples×c cell types), in which fⱼₖ denotes the unknown cell fraction of k^{th} cell type in j^{th} sample. ∥·∥, represents the L2-norm (which measures the reconstruction error) and diag( ) represents a function that extracts the diagonal entries of a matrix. The objective is to find an optimal solution for G and F with the constraint that the cell fractions (of c cell types) in any sample j sum up to 1 and all the gene expression values gᵢⱼₖ are non-negative real values. It may be assumed that the gene expression is quantified as trimmed mean of M-values (TMM) normalized TPM (transcript per million) values. The method may comprise employing a strategy introduced by Monaco et al. [8], which first estimates between-sample scaling factors upon raw TPM values using the TMM method [9], and further scaling TPM values in each sample using these scaling factors.

Problem (1) has no unique optimal solution without additional constraints and regularizations since there are more parameters to be estimated than observations [7], [10]. In one example problem (1) can be separated into two independent problems: cell fraction estimation and cell-type-specific gene expression prediction for each individual sample. In one example, the cell fraction estimation problem is formulated as follows:

$$minimize {\sum }_{i = 1}^{h} ‖ {B}_{ir}^{′} - {S}_{ir} \times {F}^{T} ‖ \\ subject to {\sum }_{k = 1}^{c} {f}_{jk} = 1 for all j , \\ {f}_{jk} \geq 0 for all j , k \qquad ( 2 )$$

where S denotes the cell-type-specific signature matrix (h genes×c cell types) and the h genes are a subset of all the m genes in G or B matrix that are preferentially over-expressed in at least one of the c cell-types and their expression is assumed to be constant across the population to arrive at an approximate solution of cell fractions in each sample. F is the same as that in equation (1), while B′ is the bulk expression matrix in equation (1) corresponding to the h genes in cell-type-specific signature matrix S. Numerous effective cell fraction estimation tools have been developed and reported to solve problem (2) [11]-[27]. The experimental analog to these methods is the cell gating procedure described in FACS. This problem may be solved using a well-known reference-based approach—CIBERSORT [24]. If needed, a batch correction approach introduced by CIBERSORTx may be applied to minimize cross-platform technical batch effects between bulk mixture profile and cell type signature profile generated from different technical platforms (e.g. bulk RNA sequencing, SmartSeq2-based single cell sequencing, 10×-based single cell sequencing and microarray expression profiling) [7]. In addition, the method may optionally comprise inputting prior known cell fractions instead of performing cell-fraction estimation de novo. Either known cell fractions or cell type signature profiles are required as input.

After F is estimated or provided, the full deconvolution problem formulated in (1) can be reduced to solving for G, given B and F. One can additionally reduce problem (1) to a simpler problem where one solves for the expected cell-type-specific expression across a group of individual samples, given the cell fractions and bulk expression matrix:

minimizeΣᵢ₌₁^{m}∥Bᵢ,.−Ēᵢ,.×F^{T}∥ (3)
subject to eᵢₖ≥0 for all i, k

where B is the same as in equation (1) and represents the input bulk expression matrix; F is also the same as that in equation (1) and denotes cell fractions; Ē is expected cell-type-specific expression matrix (m genes×c cell types) across the population, in which ēᵢₖ denotes the expected expression of gene i in cell type k. For a fixed F, a unique optimal solution for this problem exists and can be found using non-negative least squares (NNLS) [7], [28], [29]. The key difference between problem (3) and problem (1) is that the former aims to predict expected cell-type-specific expression for each gene in the population, while the latter predicts cell-type-specific expression for each gene and each sample.

One can aim to solve problem (1) approximately by making use of a greedy divide and conquer strategy that breaks down problem (1) into simpler problems (2) and (3). Newman et al, in their groundbreaking work CIBERSORTx, were the first to propose such an algorithm. In CODEFACS, we introduce the concept of confidence scores and additional algorithmic improvements. The inventors have shown that CODEFACS yields a much more accurate solution compared to CIBERSORTx in 15 benchmark datasets with ground truth data.

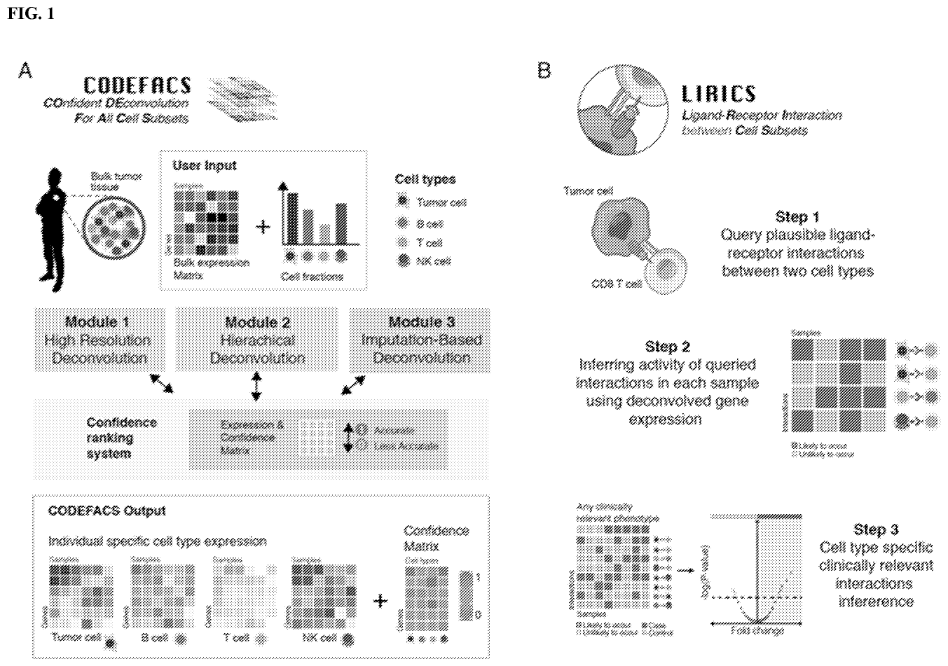

The CODEFACS algorithm comprises three modules, referred to herein as Module 1, Module 2, and Module 3, that are executed sequentially and a confidence ranking system that is invoked after the execution of each module. Module 1 comprises a high-resolution deconvolution module. First, the method described herein comprises a generalization of a two-freedom estimation method into a recursive splitting method, referred to herein as “p-freedom estimation” (the degrees of freedom represent the distinct latent sources of variability in gene expression across individuals). p-freedom estimation captures tumor heterogeneity better than the 2-freedom estimation. Second, the method described herein generalizes a previously known sliding window method by employing an ensemble of window sizes. Using an ensemble of window sizes seeks to reduce the dependence of downstream biological analyses on arbitrary choices of the window size parameter. In addition, the method described herein further comprises modules 2 and 3 (hierarchical deconvolution and imputation-based deconvolution) to overcome the shortcomings of previously known modules. The confidence ranking system uses a series of heuristics to decide where the solution can be improved by subsequent modules. A schematic of the inputs and outputs of the method described herein is shown in FIG. 1A.

### a. Confidence

The output of each module is ranked by a confidence ranking system. Each of the three prediction modules of the methods described herein operates under specific modeling assumptions that may be uniformly applicable to all genes. However, in practice, certain genes might violate these assumptions. Therefore, for such genes, one cannot confidently say whether their predicted cell-type specific expression levels closely reflect the ground truth. To systematically quantify this uncertainty, the method comprises a confidence ranking system, which can decide whether a specific prediction requires further refinement in subsequent modules by defining a ranking Φ over genes for each cell-type using confidence relevant features further described herein. Additionally, the confidence ranking system also re-evaluates the confidence level of each final prediction (gene-cell type pair) and in the end reports a confidence score between 0 and 1.

### b. Optional Steps

### (1) Cell Fractions Estimation

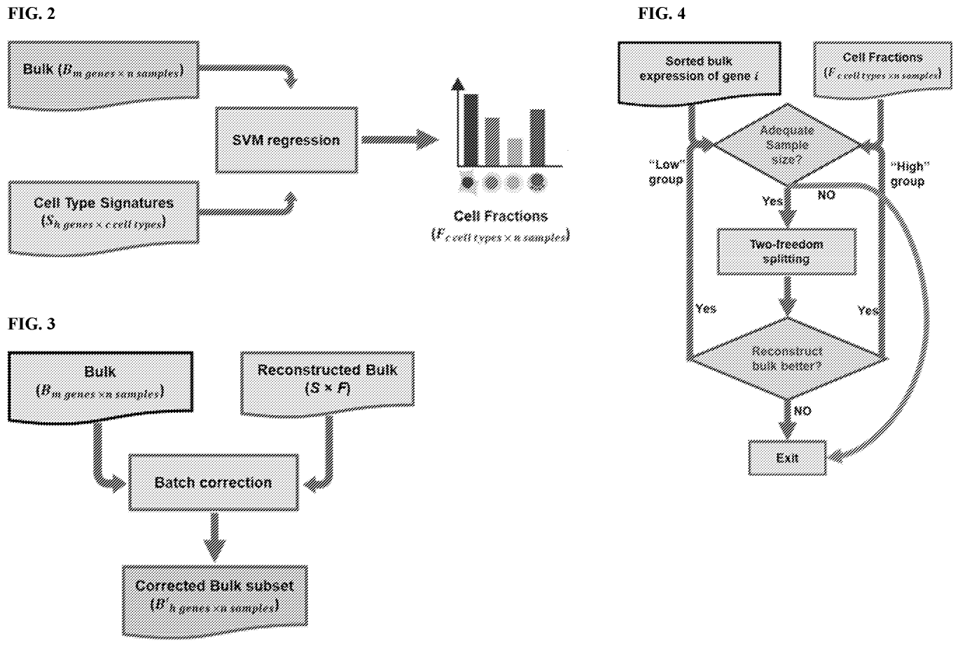

The method may optionally comprise estimating cell fractions using support vector machine (SVM)-regression according to the CIBERSORTx method (as described in Newman, A., Liu, C., Green, M. et al. Robust enumeration of cell subsets from tissue expression profiles. Nat Methods 12, 453-457 (2015), doi.org/10.1038/nmeth.3337, the contents of which are incorporated herein by reference). The method may comprise providing bulk expression and/or bulk methylation data, along with known cell-type signature profiles. Based on the bulk mixture and cell type signature profile, the SVM regression model may output a prediction of cell fraction for each cell type and sample, as set forth in FIG. 2. SVM regression need not be used if prior known cell fractions are used as input. If prior known cell fractions are provided as input, this optional step may be skipped.

### (2) Batch Correction to Refine Cell Fractions

The method may optionally comprise re-implementing a batch correction method, which may set forth in A. M. Newman et al., “Determining cell type abundance and expression from bulk tissues with digital cytometry,” Nat. Biotechnol., 2019, the contents of which are incorporated herein by reference. This account for any systematic batch effects between bulk expression and independently generated cell-type-specific signature expression data, which could bias cell-fraction estimates. The rationale for this method is that any batch effect between the given bulk expression and independently generated cell-type-specific signature expression must also be reflected in the reconstructed bulk expression using these signatures. The method may comprise further refining cell-fraction estimates of each cell-type in each sample after reducing the batch effect between the given bulk and reconstructed bulk expression of cell-type-specific signature genes (which may be performed using function ComBat( ) from the SVA package (J. T. Leek, W. E. Johnson, H. S. Parker, A. E. Jaffe, and J. D. Storey, “The SVA package for removing batch effects and other unwanted variation in high-throughput experiments,” Bioinformatics, vol. 28, no. 6, pp. 882-883, Mar. 2012, the contents of which are incorporated by reference) in R (FIG. 3). The final output of this step is a refined cell-fraction matrix F′. The method described herein comprises correcting biases among bulk RNA sequencing and SmartSeq2-based single cell sequencing datasets. This step is optional and may be skipped. The batch correction procedure may be performed as described in the section “Cross-platform normalization schemes for deconvolution” in the supplementary information of A. M. Newman et al., “Determining cell type abundance and expression from bulk tissues with digital cytometry,” Nat. Biotechnol., vol. 37, no. 7, pp. 773-782,2019, the contents of which are incorporated herein by reference.

### c. Module 1: High Resolution Deconvolution

The method described herein comprises a module (referred to herein as module 1), which comprises performing high resolution deconvolution. The deconvolution may comprise modeling observed bulk expression of a gene in a sample as the weighted sum of cell-type-specific expression of the gene from the sample, as outlined above. The high resolution deconvolution may comprise providing sorted bulk expression of each gene in the bulk gene expression data.

### (1) Determining Cell Types in which a Specific Gene is Weakly Expressed (Step 2.1)

The high resolution deconvolution may comprise determining the cell types in which a specific gene is weakly expressed. Determining if a gene i is weakly expressed in a cell type, the method may comprise performing the following statistical analysis: individuals are randomly chosen without replacement to generate 100 random subsets of individual samples and then problem (3) is solved to estimate expected cell-type-specific expression for each random subset. This bootstrapping procedure generates a distribution of expected cell-type-specific expression values ēᵢₖ in the population. The method may then comprise deriving two p-values for each cell-type k: first, an empirical p-value that is estimated by checking the percentage of solutions where ēᵢₖ>0, and second, a parametric t-test p-value. The two p-values are then combined using Fisher's method [32] to obtain a final p-value for each cell-type. If a gene is weakly expressed in a cell type (FDR≥0.2), the cell fractions of that cell type in the corresponding mixture model may be forced to be 0 to improve the deconvolution of gene expression in other cell types.

### (2) Recursively Splitting Samples into Finite Sub-Groups (Step 2.2)

High resolution deconvolution may further comprise recursively splitting samples into finite sub-groups, also referred to herein asp-freedom based splitting. With an appropriate cell-type mixture model defined for each gene, the method may comprise finding an approximate solution to problem (1). For a gene i, problem (1) may be divided into a finite number of simpler problems by assuming that individuals with similar bulk expression levels of gene i must have similar cell-type specific expression levels of gene i. All samples may be sorted in increasing order according to the bulk expression of gene i. In the two-freedom deconvolution in CIBERSORTx algorithm, one can then find a position t to partition all the sorted samples into two sorted subsets: h₁={1, 2, . . . , t−1} and h₂={t, t+1, . . . , n}, such that the expected cell-type-specific expression in each of the sub-groups (obtained from solving problem 3 using NNLS) best reconstructs the observed bulk expression. This may be performed as described in the section “Cell type expression coefficients that best explain the bulk GEP” in the supplementary information of A. M. Newman et al., “Determining cell type abundance and expression from bulk tissues with digital cytometry,” Nat. Biotechnol., vol. 37, no. 7, pp. 773-782, 2019, the contents of which are incorporated herein by reference. Either of these two sub-groups can now be recursively partitioned further into smaller sub-groups in a similar fashion if the re-construction error keeps dropping and the sub-group sample size stays above 1.9 times the number of cell-types. This is referred to as a p-freedom approach (FIG. 4).

In one example, the recursive splitting pseudo-code is:

```
recursive_splitting( ):
Input:
  F = cell fraction matrix
  B = bulk expression matrix
  size = the number of samples requiredfor NNLS
  i = gene index
  st = start position in the sorted list of samples
  ed = end position in the sorted list of samples
Output:
  NULL
Function:
Ēᵢᵣ = expected cell-type specific expression of gene i over all sorted samples in the range
{st, ..., ed} (Obtained from solving problem 3)
if (ed − st + 1 < 2 × size) {
    save Ēᵢᵣ;
    exit;
}
else {
  t = 2-freedom index that splits sorted samples in the range {st, .... ed}, into two
  subsets: {st, ..., st + t} and {st + t + 1, ..., ed} (See CIBERSORTx algorithm for
  more details)
  L̅ᵢᵣ = expected cell-type specific expression of gene i over all sorted samples in
  the range {st, ..., st + t} (Obtainedfrom solving problem 3)
  H̅ᵢᵣ = expected cell-type specific expression of gene i over all sorted samples in
  the range {st + t + 1, ..., ed} (Obtainedfrom solving problem 3)
  Err1 = ||Bᵢ,_{(st,...,ed)} − (Ēᵢᵣ × (F_{(st,...,ed)},.)^{T})||
  Err2 = ||Bᵢ,_{(st,...,ed)} − [(L̅ᵢᵣ × (F_{(st,...,st+t)},.)^{T}), (H̅ᵢᵣ × (F_{(st+t+1,...,ed)},.)^{T})]||
  If(Err2 < Err1){
    Record the splitting results;
    Call: recursive_splitting(F,B,size,i,st, st+t);
    Call: recursive_splitting(F,B, size, I,st+t+1, ed);
    Exit;
  }
  Exit;
}
End
```

### (3) Ensemble Sliding Window Deconvolution (Step 2.3)

The high resolution deconvolution may comprise performing ensemble sliding window deconvolution. In one example, for gene i, a sliding window is defined over the sorted list of samples with a specific window size s. For each window of sorted samples, problem (3) may be solved using NNLS to estimate the expected cell-type-specific expression within that window. Cell-type-specific expression for each individual may then approximated by re-distributing population-level estimates of cell-type specific expression within each sliding window (based on the assumption that subsets of individuals with very similar bulk expression profiles have a shared cell-type-specific expression profile, and may be described in the CIBERSORTx algorithm referenced herein). Thereafter, the initial approximate predictions from the sliding window deconvolution of window size s are refined using a linear-regression-based smoothing procedure such that the distribution of expression values is statistically consistent with population level estimates from the p-freedom estimation step. This is based on the assumption that the estimated distribution of cell-type specific expression in each subset is robust to outliers.

Ensemble sliding window deconvolution may comprise setting up window sizes ranging from s₁=˜1.9 times the number of cell types to

$${s}_{t} = max ( 4 \times number of cell types , \frac{sample size}{2} )$$

and then performing the above sliding window deconvolution for each of these window sizes. Given multiple solutions for the cell-type-specific expression profile of each sample derived from multiple choices of sliding-window sizes (S₁ to sₜ), their average can be computed to obtain a single initial approximate solution to problem (1) (referred to as ensemble of window sizes); steps 2.1-2.3 are repeated for the next gene until all the genes are done.

In one example, the ensemble sliding window pseudo code is:

```
ensemble_sliding_window( ):
Input:
B = bulk expression matrix
F = cell fraction matrix
i = gene index
n = the number of samples
Gₚ = predicted expression in p-splitting deconvolution
c = the number of cell type;
m = the number of genes;
Output:
    Cell-type-specific expression matrix (3-dimension) for all samples: G
Function:
    ${s}_{1} = \frac{( 1.5 \times ct )}{0.8} ;$
    ${s}_{t} = max ( 4 c , round ( \frac{size}{2} ) ) ;$
    G = initialize_matrix(num × size × ct, 0);
    for iteration w from s₁ to sₜ
    {
        G = G + sliding_window(B, F, Gₚ, w, i);
    }
    $G = \frac{G}{( {s}_{t} - {s}_{1} + 1 )} ;$
Return G;
End
```

### (4) Confidence Ranking for the Predictions Following Module 1

Genes that follow the modeling assumptions of module 1 may be more likely to have their cell-type-specific expression levels predicted confidently. Hence, while executing module 1, the method may comprise collecting a series of features that could be useful in determining confidence level of expression predictions for each gene-cell-type pair. These include one or more of: p-value of t-test determining if a gene is weakly expressed in a cell-type (obtained from completion of step 2.1), ratio of mean predicted expression levels and p-value of differential expression between subgroups sg₁ and sg₂ (obtained from completion of steps 2.2 and 2.3), Spearman correlation between predicted cell-type-specific expression and bulk gene expression across samples, and Spearman correlation between bulk expression and the cell fraction across samples etc. The method may then comprise defining a ranking Φ using this feature space such that genes achieving a high rank are on average ranked highly by each feature as follows:

$$Φ ( gene i , celltype k ) = \frac{{\sum }_{feature ∈ set} rank ( feature ( gene i , celltype k ) )}{\vert set \vert}$$

where Φ (gene i, celltype k) represents the prediction rank of gene i in cell type k, feature represents each feature we collected in the feature set, |set| represents the number of feature types and feature (gene i, celltype k) denotes the each feature vector of gene i in cell type k.

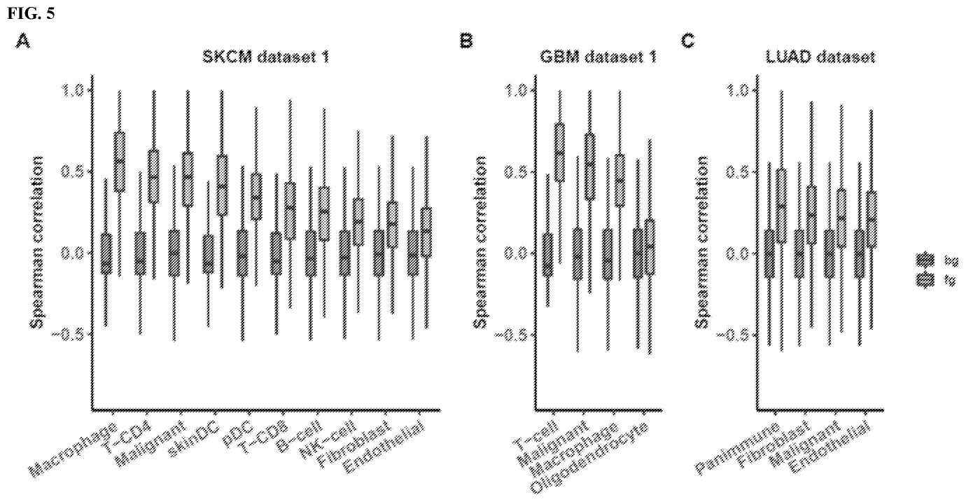

Additionally, it is well known that (the proteins encoded by) genes may interact with each other and behave collaboratively as complexes [33]; also, gene regulation is highly dependent on numerous regulatory elements including transcription factors [34], [35]. When looking at single cell expression data from three independent single cell datasets, the inventors have found that expression profiles of 1000 randomly selected genes within the same cell type are much more strongly correlated than expected by random chance (FIG. 5). Hence, genes whose expression was confidently predicted in each cell-type may have correlated predictions. Therefore, ranking Φ may be updated to Φ′ by accounting for these correlations as follows:

$${Φ}^{′} ( gene i , celltype k ) ∼ {\sum }_{j ∈ Q} Φ ( gene j , celltype k ) ,$$

where Q represents the set of genes whose predicted expression in cell-type k is strongly correlated with the predicted expression of gene i in cell-type k (Spearman correlation≥0.4). Described below is how the confidence ranking system takes this ranking Φ′ and decides which genes need to be passed to module 2 for each cell-type.

For each cell type k, two sets may be defined: [symbol] ₖ and [symbol] , which are called the “high” and “low”-confidence sets of cell-type k. Genes belonging to the set [symbol] will be passed on to module 2. Let mₖ be the number of genes whose predicted expression distribution in the population is at least bi-modal (i.e fold change in expression between the subsets h₁ and h₂>1). Genes are then assigned to the high, low confidence set of each cell-type by the confidence ranking system using the following rule:

Add gene i to set:

$$, if {Φ}^{′} ( gene i , celltype k ) < round ( \frac{{m}_{k}^{2}}{2 m} ) \\ {ℒ}_{k} , if {Φ}^{′} ( gene i , celltype k ) > m - round ( \frac{{m}_{k}}{2} \times ( 1 - \frac{{m}_{k}}{m} ) )$$

The results of the assignment may be stored in a confidence matrix C (m genes×c celltypes) encoding the high vs low confidence memberships of each gene in each cell type.

### d. Module 2—Hierarchical Deconvolution for Low-Confidence Genes Emerging from Step 3

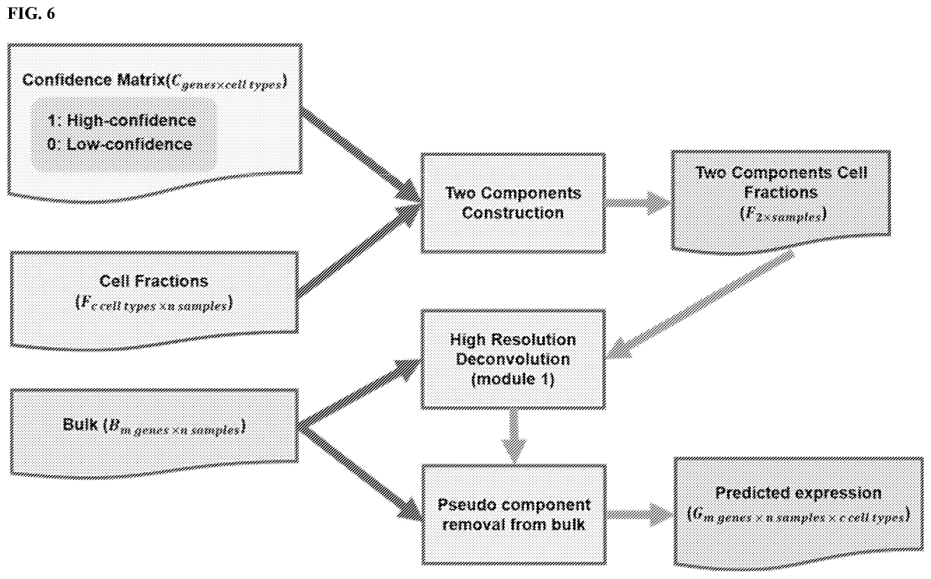

The method further comprises a module (referred to herein as module 2), which may comprise simplifying the general cell-type mixture model described in module 1 to a 2-component mixture model (FIG. 6). Specifically, hierarchical deconvolution may comprise, for a gene i in the low-confidence set of cell type k, modeling its observed bulk expression level in a sample as a mixture of 2 components: the first component represents the cell-type k and the second component represents a pseudo-cell-type that is a composite of all the cell-types except k^{th} cell type. Module 1 may then be re-run to predict individual specific expression of gene i for the pseudo cell-type. Finally, the prediction for the pseudo cell-type is subtracted from the bulk to approximately re-estimate the individual specific expression of gene i in cell type k. This is based on the assumption that the expression of the pseudo component might be better predicted than the expression of cell-type k using module 1, especially if cell type k is not abundant or gene i is weakly expressed in cell type k. The above steps are repeated for all the remaining genes in the low-confidence set of each cell-type. In one example, the hierarchical deconvolution pseudo code is:

```
hierarchical_deconvolution( ):
Input:
B = bulk expression matrix
F = cell fraction matrix
C = confidence level matrix
G = global variable for estimated gene expression across cell types
and samples
c = the number of cell types;
m = the number of genes;
Output:
    NULL
Function:
    for iteration k from 1 to c
    { [symbol]
          = [F[k, ], 1 − F[k, ]]
for iteration d from 1 to m
        {
            if (C[i, k] ∈ [symbol]  ₖ )
            {
G^{pseudo} = High_resolution_deconvolution(B, [symbol]  , i);
                $G [ i , , k ] = \frac{B [ \text{?} , ] - \text{?} [ 2 , ] * {G}^{pseudo} [ \text{?} , , 2 ]}{\text{?} [ 1 , ]}$
            }
        }
    }
End [symbol]
indicates data missing or illegible when filed
```

### e. Confidence Ranking of Predictions Emerging from Module 2

Following module 2, the following ranking Φ for genes in the low confidence set of each cell type may be defined as follows:

$$For gene i ∈ {k}_{} , \\ Φ ( gene i , celltype k ) ∼ \frac{1}{\vert \vert} {\sum }_{j ∈} ρ ( new predictions for gene i , predictions for gene j )$$

where ρ represents the spearman correlation, | [symbol] ₖ| represents the number of genes in high confidence set [symbol] ₖ. This is again based on observations of single cell expression data described above from which we deduce that genes with similar confidence levels are expected to have correlated predictions (FIG. 5). The confidence ranking system may be as described below, which may take this ranking of genes in the low confidence set of each cell-type and decide which genes need to be upgraded to the high confidence set:

Let | [symbol] | be the number of genes in the low confidence set of cell-type k, N be the total number of genes and CFMₖ be the mean cell fraction of cell-type k. The confidence ranking system upgrades the membership of genes from the low confidence set [symbol] to the high confidence set [symbol] ₖ using the following rule:

$$For gene i ∈ {k}_{} , \\ = ⋃ \{ i \} , if Φ ( gene i , celltype k ) < round ( \frac{{\vert {ℒ}_{k} \vert}^{2} * {CFM}_{k}}{2 m} ) \\ {ℒ}_{k} = {ℒ}_{k} \backslash \{ i \} , if Φ ( gene i , celltype k ) > m - round ( \frac{{\vert {ℒ}_{k} \vert}^{2}}{2 m} )$$

The results of the assignment are stored in the confidence matrix C (m genes×c celltypes) encoding the high vs low confidence memberships of each gene in each cell-type.

### f. Module 3—Imputation-Based Deconvolution for Low-Confidence Genes Emerging from Step 5

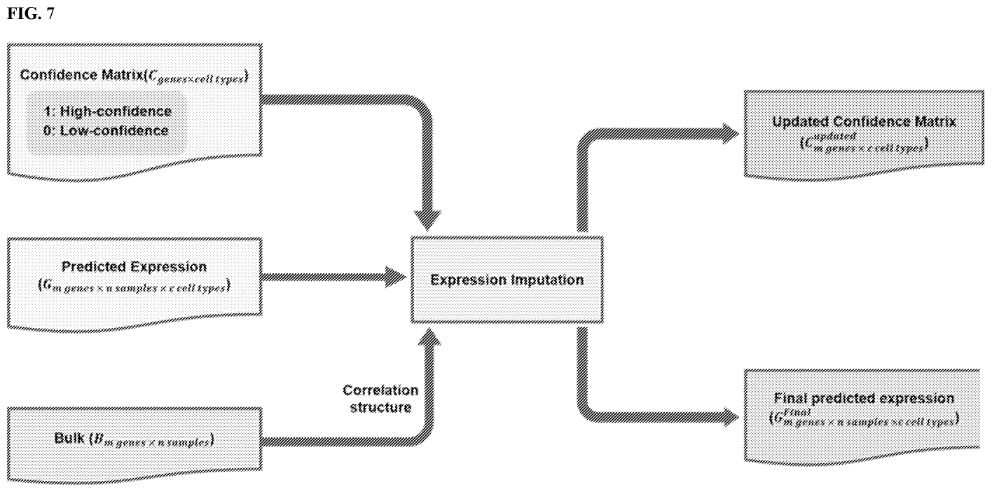

The method may further comprise a module (referred to herein as module 3), which operates on the assumption that the expression levels of two genes are supposed to be correlated in some cell types if we observe that their bulk expression is significantly correlated [8]. For a gene i still in the low-confidence set of cell-type k, the Spearman correlations between the bulk expression profile of gene i and bulk expression profiles of genes in the high confidence set of cell-type k are estimated. If the bulk expression profile of gene i is highly correlated (Spearman correlation≥0.5) with the bulk expression profiles of more than two genes in the high-confidence set of cell-type k, a lasso regression-based machine learning model is trained using bulk expression to impute individual specific expression of gene i in cell type k based on predicted expression profiles of high-confidence genes in cell-type k (FIG. 7). The above steps are repeated for all the remaining genes in the low-confidence set of each cell-type. In one example, imputation-based deconvolution comprises the following:

```
Imputation_based_deconvolution( ):
Input:
B = bulk expression matrix
F = cell fraction matrix
C = confidence level matrix
G = global variable for estimated gene expression across cell types and samples
c = the number of cell types;
Output:
    NULL
Function:
    for iteration k from 1 to c
    {
      Conf_{high} = all genes ∈ [symbol] ₖ;
      Conf_{low} = all genes ∈ [symbol] ₖ;
  Corrs = pairwise Spearman correlation matrix (B[Conf_{low}, ] × B[Conf_{high}, ]);
      for each gene i ∈ [symbol] ₖ
        {
            if (sum(Corrs[i, ] ≥ 0.5) ≥ 2)
          {
              Train imputation model: B[i, ] ~ fᵢₘₚ(B[Conf_{high},]);
              Impute G[i, , k] = fᵢₘₚ(G[Conf_{high},, k]);
          }
        }
    }
End
```

### g. Confidence Ranking for Predictions Emerging from Module 3

The method may further comprise applying a confidence ranking system to the output of module 3. Following module 3, the following confidence ranking features for each gene i in the low confidence set of cell-type k may be collected: the correlations of predicted gene expression with bulk expression, the correlation between cell fractions and bulk expression, number of genes as features in the imputation model, average spearman correlation between new predictions of gene i and predictions of genes in the high confidence set of cell-type k. As before, we define ranking Φ using this feature space such that genes achieving a high rank are on average ranked highly by each feature. Genes that are ranked among top 80% of all genes in the low confidence set of a cell-type k are now upgraded to the high confidence set of cell-type k by the confidence ranking system.

### h. Final Output—Confidence Scores and Cell-Type-Specific Gene Expression Profiles of Each Sample

The method further comprises generating a final confidence score for each pair of gene and specific cell type. To transform high- vs low-confidence set memberships of genes in each cell-type (which were based on artificially defined rules/cut-offs for easy implementation of the greedy algorithm), into scores that are continuous in the range [0,1], the following final steps may be taken: (a) The pair-wise correlations between the predicted expression profile of a gene i in cell-type k and predicted expression profiles of genes belonging to the high-confidence set of cell-type k are averaged to generate a score for gene i; (b) the cell-type-specific expression predictions across the samples (columns) are randomly shuffled to generate a background and step a is repeated to estimate a background distribution of scores for each gene; (c) for each gene and each cell type, one can now determine an empirical p-value pv based on this background distribution of scores. These p-values quantify the probability of a gene having high confidence predictions by random chance if its predictions are correlated with predictions of any other genes belonging to the high-confidence set of a cell-type. The p-values are low for genes that are part of the high confidence set and high for genes part of low confidence set and intermediate for genes belonging to neither. Hence, 1-pv is recorded as the final confidence score for each gene-cell-type pair. The final outputs of CODEFACS are the approximate solution for 3-dimensional matrix G after execution of module 3 (imputation-based deconvolution) and confidence scores for each gene-cell-type pair.

### 2. LIRICS Algorithm

Also provided herein is a method of identifying ligand-receptor interactions between two cell types, which may be a first cell type and a second cell type. A schematic of the method is outlined in FIG. 1B.

### a. Curation of Established Ligand-Receptor Protein-Protein Interactions Between Cell Types in the Tissue Microenvironment

The method may comprise providing a catalog of ligand-receptor interactions among a plurality of cell types, and the expectation distribution of ligands and receptor on each cell type. In one example, the catalog comprises known protein-protein interactions between tumor, epithelial, immune and stromal cell types in the tissue microenvironment, and may comprise all such protein-protein interactions. Specifically, the interactions correspond to one or more of cytokine/chemokine-cytokine/chemokine receptor interactions, ligand-receptor interactions involved in cell adhesion/leukocyte trans-endothelial migration, ligand-receptor interactions involving the TNF receptor superfamily and lastly, ligand receptor interactions involved in regulation of NK and T cell cytotoxicity were all merged into one excel spreadsheet [36]-[43]. In one example, the catalog comprises 348 established ligand-receptor interactions, as shown in Table 1. This catalog primarily covers proteins that have well characterized immunological functions. Certain receptors are complexes encoded by more than one gene, such as TGF beta family receptors. Furthermore, certain proteins serve as both ligands on some cell types and receptors on other cell types, such as HVEM (TNFRSF14).

TABLE 1

| Ligand | Receptor | Annotations |
|---|---|---|
| CCL1 | CCR8 | chemotaxis |
| CCL1 | CCR11 | chemotaxis |
| CCL25 | CCR9 | chemotaxis |
| CCL25 | CCR11 | chemotaxis |
| CCL19 | CCR11 | chemotaxis |
| CCL21 | CCR7 | chemotaxis |
| CCL3L1 | CCR5 | chemotaxis |
| CCL4 | CCR5 | chemotaxis |
| CCL4L1 | CCR5 | chemotaxis |
| CCL4L2 | CCR5 | chemotaxis |
| CCL17 | CCR4 | chemotaxis |
| CCL22 | CCR4 | chemotaxis |
| CCL5 | CCR5 | chemotaxis |
| CCL5 | CCR3 | chemotaxis |
| CCL3 | CCR5 | chemotaxis |
| CCL3 | CCR1 | chemotaxis |
| CCL9 | CCR1 | chemotaxis |
| CCL14 | CCR1 | chemotaxis |
| CCL16 | CCR1 | chemotaxis |
| CCL23 | CCR1 | chemotaxis |
| CCL15 | CCR3 | chemotaxis |
| CCL15 | CCR1 | chemotaxis |
| CCL13 | CCR1 | chemotaxis |
| CCL13 | CCR3 | chemotaxis |
| CCL13 | CCR11 | chemotaxis |
| CCL13 | CCR2 | chemotaxis |
| CCL6 | CCR1 | chemotaxis |
| CCL6 | CCR3 | chemotaxis |
| CCL6 | CCR2 | chemotaxis |
| CCL7 | CCR1 | chemotaxis |
| CCL7 | CCR3 | chemotaxis |
| CCL7 | CCR2 | chemotaxis |
| CCL2 | CCR2 | chemotaxis |
| CCL12 | CCR2 | chemotaxis |
| CCL11 | CCR3 | chemotaxis |
| CCL24 | CCR3 | chemotaxis |
| CCL26 | CCR3 | chemotaxis |
| CCL27 | CCR2 | chemotaxis |
| CCL27 | CCR10 | chemotaxis |
| CCL28 | CCR3 | chemotaxis |
| CCL28 | CCR10 | chemotaxis |
| CCL20 | CCR6 | chemotaxis |
| CXCL1 | CXCR1 | chemotaxis |
| CXCL5 | CXCR1 | chemotaxis |
| CXCL6 | CXCR1 | chemotaxis |
| CXCL8 | CXCR1 | chemotaxis |
| CXCL1 | CXCR2 | chemotaxis |
| CXCL5 | CXCR2 | chemotaxis |
| CXCL6 | CXCR2 | chemotaxis |
| CXCL8 | CXCR2 | chemotaxis |
| CXCL2 | CXCR2 | chemotaxis |
| CXCL3 | CXCR2 | chemotaxis |
| CXCL7 | CXCR2 | chemotaxis |
| CXCL4 | CXCR3 | chemotaxis |
| CXCL4L1 | CXCR3 | chemotaxis |
| CXCL9 | CXCR3 | chemotaxis |
| CXCL10 | CXCR3 | chemotaxis |
| CXCL11 | CXCR3 | chemotaxis |
| CXCL13 | CXCR3 | chemotaxis |
| CXCL13 | CXCR5 | chemotaxis |
| CXCL12 | CXCR4 | chemotaxis |
| CXCL12 | CXCR7 | chemotaxis |
| CXCL16 | CXCR6 | chemotaxis |
| XCL1 | XCR1 | |
| XCL2 | XCR1 | |
| CX3CL1 | CX3CR1 | chemotaxis |
| IL2 | IL2RA; IL2RB; | activating/co-stimulatory |
| | IL2RG | |
| IL2 | IL2RB; IL2RG | activating/co-stimulatory |
| IL4 | IL4R; IL2RG | |
| IL7 | IL7R; IL2RG | |
| IL9 | IL9R; IL2RG | |
| IL15 | IL15RA | activating/co-stimulatory |
| IL21 | IL21R; IL2RG | |
| TSLP | IL7R; TSLPR | |
| IL3 | IL3RA; CSF2RB | |
| IL5 | IL5RA; CSF2RB | |
| CSF2 | CSF2RA; CSF2RB | |
| IL4 | IL4R | |
| IL4 | IL4R; IL13RA1 | |
| IL4 | IL4R; IL13RA2 | |
| IL13 | IL4R; IL13RA2 | |
| IL13 | IL13RA1 | |
| IL13 | IL13RA2 | |
| EPO | EPOR | |
| GH1 | GHR | |
| GH2 | GHR | |
| PRL | PRLR | |
| TPO | MPL | |
| CSF3 | CSF3R | |
| LEP | LEPR | |
| IL6 | IL6R; IL6ST | pro-inflammatory |
| IL11 | IL11RA; IL6ST | |
| IL12 | IL12RB1; IL12RB2 | pro-inflammatory |
| IL23A | IL23R; IL12RB1 | |
| IL27A | IL27RA; IL6ST | |
| IL31 | IL31RA; OSMR | |
| CLCF1 | CNTFR; LIFR; IL6ST | |
| CNTF | CNTFR; LIFR; IL6ST | |
| LIF | LIFR; IL6ST | |
| OSM | LIFR; IL6ST | |
| IL10 | IL10RA; IL10RB | |
| IL19 | IL20RA; IL20RB | |
| IL20 | IL20RA; IL20RB | |
| IL20 | IL22RA1; IL20RB | |
| IL24 | IL20RA; IL20RB | |
| IL24 | IL22RA1; IL20RB | |
| IL22 | IL22RA1; IL10RB | |
| IL26 | IL20RA; IL10RB | |
| IL28A | IL28RA; IL10RB | |
| IL28B | IL28RA; IL10RB | |
| IL29 | IL28RA; IL10RB | |
| IFNA | IFNAR1; IFNAR2 | pro-inflammatory |
| IFNBI | IFNAR1; IFNAR2 | pro-inflammatory |
| IFNW1 | IFNAR1; IFNAR2 | pro-inflammatory |
| IFNK | IFNAR1; IFNAR2 | pro-inflammatory |
| IFNT1 | IFNAR1; IFNAR2 | pro-inflammatory |
| IFNG | IFNGR1; IFNGR2 | pro-inflammatory |
| IL1A | IL1R1; IL1RAP | pro-inflammatory |
| IL1B | IL1R1; IL1RAP | pro-inflammatory |
| IL1RN | IL1R1; IL1RAP | pro-inflammatory |
| IL1A | IL1R2 | pro-inflammatory |
| IL1B | IL1R2 | pro-inflammatory |
| IL1RN | IL1R2 | pro-inflammatory |
| IL1F5 | IL1RL2; IL1RAP | pro-inflammatory |
| IL1F6 | IL1RL2; IL1RAP | pro-inflammatory |
| IL1F8 | IL1RL2; IL1RAP | pro-inflammatory |
| IL1F9 | IL1RL2; IL1RAP | pro-inflammatory |
| IL1F10 | IL1RL2; IL1RAP | pro-inflammatory |
| IL1F7 | IL18R1; IL18RAP | pro-inflammatory |
| IL18 | IL18R1; IL18RAP | pro-inflammatory |
| IL33 | ST2; IL1RAP | |
| IL17A | IL17RA; IL17RC | |
| IL17F | IL17RA; IL17RC | |
| IL17B | IL17RB | |
| IL17C | IL17RE | |
| IL17E | IL17RA; IL17RB | |
| IL16 | CD4 | |
| IL34 | CSF1R | |
| CSF1 | CSF1R | |
| TNF | TNFR1 | pro-inflammatory |
| TNF | TNFR2 | pro-inflammatory |
| LTA | TNFR1 | |
| LTA | TNFR2 | |
| LTA | LTBR | |
| LTA | TNFRSF14 | activating/co-stimulatory |
| LTB | LTBR | |
| TNFSF14 | LTBR | activating/co-stimulatory |
| TNFSF14 | TNFRSF14 | activating/co-stimulatory |
| TNFSF14 | TNFRSF6B | |
| FASLG | TNFRSF6B | |
| FASLG | FAS | |
| TNFSF15 | TNFRSF6B | |
| TNFSF15 | TNFRSF25 | activating/co-stimulatory |
| TNFSF10 | TNFRSF10A | |
| TNFSF10 | TNFRSF10B | |
| TNFSF10 | TNFRSF10C | |
| TNFSF10 | TNFRSF10D | |
| EDA | EDAR | |
| EDA | EDA2R | |
| NGF | NGFR | |
| TNFSF11 | TNFRSF11A | |
| TNFSF11 | TNFRSF11B | |
| TNFSF12 | TNFRSF12A | |
| CD70 | CD27 | activating/co-stimulatory |
| TNFSF8 | TNFRSF8 | activating/co-stimulatory |
| CD40LG | CD40 | activating/co-stimulatory |
| TNFSF9 | TNFRSF9 | activating/co-stimulatory |
| TNFSF4 | TNFRSF4 | activating/co-stimulatory |
| TNFSF18 | TNFRSF18 | activating/co-stimulatory |
| TNFSF13 | TNFRSF17 | activating/co-stimulatory |
| TNFSF13 | TNFRSF13B | activating/co-stimulatory |
| TNFSF13B | TNFRSF17 | activating/co-stimulatory |
| TNFSF13B | TNFRSF13B | activating/co-stimulatory |
| TNFSF13B | TNFRSF13C | activating/co-stimulatory |
| TGFB1 | TGFBR1; TGFBR2 | |
| TGFB2 | TGFBR1; TGFBR2 | |
| TGFB3 | TGFBR1; TGFBR2 | |
| TGFB1 | ACVRL1; TGFBR2 | |
| TGFB2 | ACVRL1; TGFBR2 | |
| TGFB3 | ACVRL1; TGFBR2 | |
| GDF2 | ACVRL1; ACVR2A | |
| GDF2 | ACVRL1; BMPR2 | |
| BMP10 | ACVRL1; ACVR2A | |
| BMP10 | ACVRL1; BMPR2 | |
| INHA | ACVR2A | |
| INHA | ACVR2B | |
| BMP3 | ACVR2B | |
| GDF10 | ACVR1B; ACVR2A | |
| GDF11 | TGFBR1; ACVR2A | |
| GDF11 | TGFBR1; ACVR2B | |
| GDF11 | ACVR1B; ACVR2A | |
| GDF11 | ACVR1B; ACVR2B | |
| MSTN | TGFBR1; ACVR2B | |
| MSTN | ACVR1B; ACVR2B | |
| INHBA | ACVR1B; ACVR2A | |
| INHBA | ACVR1B; ACVR2B | |
| INHBB | ACVR1B; ACVR2A | |
| INHBB | ACVR1B; ACVR2B | |
| INHBB | ACVR1C; ACVR2A | |
| INHBB | ACVR1C; ACVR2B | |
| GDF1 | ACVR1B; ACVR2A | |
| GDF1 | ACVR1B; ACVR2B | |
| GDF1 | ACVR1C; ACVR2A | |
| GDF1 | ACVR1C; ACVR2B | |
| GDF3 | ACVR1B; ACVR2A | |
| GDF3 | ACVR1B; ACVR2B | |
| GDF3 | ACVR1C; ACVR2A | |
| GDF3 | ACVR1C; ACVR2B | |
| NODAL | ACVR1B; ACVR2A | |
| NODAL | ACVR1B; ACVR2B | |
| NODAL | ACVR1C; ACVR2A | |
| NODAL | ACVR1C; ACVR2B | |
| GDF9 | ACVR1B; BMPR2 | |
| AMH | ACVR1; AMHR2 | |
| AMH | BMPR1A; AMHR2 | |
| BMP2 | BMPR1A; ACVR2A | |
| BMP2 | BMPR1A; ACVR2B | |
| BMP2 | BMPR1A; BMPR2 | |
| BMP2 | BMPR1B; ACVR2A | |
| BMP2 | BMPR1B; ACVR2B | |
| BMP2 | BMPR1B; BMPR2 | |
| BMP4 | BMPR1A; ACVR2A | |
| BMP4 | BMPR1A; ACVR2B | |
| BMP4 | BMPR1A; BMPR2 | |
| BMP4 | BMPR1B; ACVR2A | |
| BMP4 | BMPR1B; ACVR2B | |
| BMP4 | BMPR1B; BMPR2 | |
| GDF5 | BMPR1A; ACVR2A | |
| GDF5 | BMPR1A; ACVR2B | |
| GDF5 | BMPR1A; BMPR2 | |
| GDF5 | BMPR1B; ACVR2A | |
| GDF5 | BMPR1B; ACVR2B | |
| GDF5 | BMPR1B; BMPR2 | |
| GDF6 | BMPR1A; ACVR2A | |
| GDF6 | BMPR1A; ACVR2B | |
| GDF6 | BMPR1A; BMPR2 | |
| GDF6 | BMPR1B; ACVR2A | |
| GDF6 | BMPR1B; ACVR2B | |
| GDF6 | BMPR1B; BMPR2 | |
| GDF7 | BMPR1A; ACVR2A | |
| GDF7 | BMPR1A; ACVR2B | |
| GDF7 | BMPR1A; BMPR2 | |
| GDF7 | BMPR1B; ACVR2A | |
| GDF7 | BMPR1B; ACVR2B | |
| GDF7 | BMPR1B; BMPR2 | |
| BMP15 | BMPR1B; BMPR2 | |
| BMP5 | AVCR1; AVCR2A | |
| BMP5 | AVCR1; AVCR2B | |
| BMP5 | AVCR1; BMPR2 | |
| BMP5 | BMPR1A; AVCR2A | |
| BMP5 | BMPR1A; AVCR2B | |
| BMP5 | BMPR1A; BMPR2 | |
| BMP5 | BMPR1B; AVCR2A | |
| BMP5 | BMPR1B; AVCR2B | |
| BMP5 | BMPR1B; BMPR2 | |
| BMP6 | AVCR1; AVCR2A | |
| BMP6 | AVCR1; AVCR2B | |
| BMP6 | AVCR1; BMPR2 | |
| BMP6 | BMPR1A; AVCR2A | |
| BMP6 | BMPR1A; AVCR2B | |
| BMP6 | BMPR1A; BMPR2 | |
| BMP6 | BMPR1B; AVCR2A | |
| BMP6 | BMPR1B; AVCR2B | |
| BMP6 | BMPR1B; BMPR2 | |
| BMP7 | AVCR1; AVCR2A | |
| BMP7 | AVCR1; AVCR2B | |
| BMP7 | AVCR1; BMPR2 | |
| BMP7 | BMPR1A; AVCR2A | |
| BMP7 | BMPR1A; AVCR2B | |
| BMP7 | BMPR1A; BMPR2 | |
| BMP7 | BMPR1B; AVCR2A | |
| BMP7 | BMPR1B; AVCR2B | |
| BMP7 | BMPR1B; BMPR2 | |
| BMP8 | AVCR1; AVCR2A | |
| BMP8 | AVCR1; AVCR2B | |
| BMP8 | AVCR1; BMPR2 | |
| BMP8 | BMPR1A; AVCR2A | |
| BMP8 | BMPR1A; AVCR2B | |
| BMP8 | BMPR1A; BMPR2 | |
| BMP8 | BMPR1B; AVCR2A | |
| BMP8 | BMPR1B; AVCR2B | |
| BMP8 | BMPR1B; BMPR2 | |
| HLA-A | KIR2DL2 | checkpoint/inhibitory |
| HLA-A | KIR2DL3 | checkpoint/inhibitory |
| HLA-A | KIR3DL1 | checkpoint/inhibitory |
| HLA-A | KIR3DL2 | checkpoint/inhibitory |
| HLA-A | LILRB1 | checkpoint/inhibitory |
| HLA-A | LILRB2 | checkpoint/inhibitory |
| HLA-B | KIR2DL2 | checkpoint/inhibitory |
| HLA-B | KIR2DL3 | checkpoint/inhibitory |
| HLA-B | KIR3DL1 | checkpoint/inhibitory |
| HLA-B | KIR3DL2 | checkpoint/inhibitory |
| HLA-B | LILRB1 | checkpoint/inhibitory |
| HLA-B | LILRB2 | checkpoint/inhibitory |
| HLA-C | KIR2DL1 | checkpoint/inhibitory |
| HLA-C | KIR2DL2 | checkpoint/inhibitory |
| HLA-C | KIR2DL3 | checkpoint/inhibitory |
| HLA-C | KIR2DS4 | activating/co-stimulatory |
| HLA-C | KIR2DS3 | activating/co-stimulatory |
| HLA-C | KIR2DS2 | activating/co-stimulatory |
| HLA-C | KIR2DS1 | activating/co-stimulatory |
| HLA-C | LILRA3 | activating/co-stimulatory |
| HLA-C | LILRB1 | checkpoint/inhibitory |
| HLA-C | LILRB2 | checkpoint/inhibitory |
| HLA-E | KLRC1; KLRD1 | checkpoint/inhibitory |
| HLA-E | KLRC2; KLRD1 | activating/co-stimulatory |
| HLA-E | KLRC3; KLRD1 | activating/co-stimulatory |
| HLA-G | KIR2DL4 | checkpoint/inhibitory |
| HLA-G | KIR3DL2 | checkpoint/inhibitory |
| HLA-G | LILRB1 | checkpoint/inhibitory |
| HLA-G | LILRB2 | checkpoint/inhibitory |
| HLA-F | KIR3DL1 | checkpoint/inhibitory |
| HLA-F | KIR3DL2 | checkpoint/inhibitory |
| HLA-F | KIR3DS1 | activating/co-stimulatory |
| HLA-F | LILRB1 | checkpoint/inhibitory |
| HLA-F | LILRB2 | checkpoint/inhibitory |
| MICA | KLRK1 | activating/co-stimulatory |
| MICB | KLRK1 | activating/co-stimulatory |
| ULBP1 | KLRK1 | activating/co-stimulatory |
| ULBP2 | KLRK1 | activating/co-stimulatory |
| ULBP3 | KLRK1 | activating/co-stimulatory |
| HA | NCR1 | activating/co-stimulatory |
| CD48 | CD244 | activating/co-stimulatory |
| ICAM1 | ITGAL; ITGB2 | |
| ICAM2 | ITGAL; ITGB2 | |
| ICOSLG | ICOS | activating/co-stimulatory |
| PVR | TIGIT | checkpoint/inhibitory |
| NECTIN2 | TIGIT | checkpoint/inhibitory |
| NECTIN3 | TIGIT | checkpoint/inhibitory |
| CD86 | CTLA4 | checkpoint/inhibitory |
| CD80 | CTLA4 | checkpoint/inhibitory |
| CD86 | CD28 | activating/co-stimulatory |
| CD80 | CD28 | activating/co-stimulatory |
| PDCD1LG2 | PDCD1 | checkpoint/inhibitory |
| CD274 | PDCD1 | checkpoint/inhibitory |
| LGALS9 | HAVCR2 | checkpoint/inhibitory |
| ICOSLG | CD28 | activating/co-stimulatory |
| TIMD4 | HAVCR1 | activating/co-stimulatory |
| CD48 | CD2 | activating/co-stimulatory |
| CD58 | CD2 | activating/co-stimulatory |
| PVR | CD226 | activating/co-stimulatory |
| NECTIN2 | CD226 | activating/co-stimulatory |
| CD274 | CD80 | checkpoint/inhibitory |
| TNFRSF14 | CD160 | |
| CD200 | CD200R1 | checkpoint/inhibitory |
| ALCAM | CD6 | activating/co-stimulatory |
| TNFRSF14 | BTLA | checkpoint/inhibitory |

### b. Expected Distribution of Ligands and Receptors Across Different Cell-Types from Prior Knowledge

The database may assign a binary indicator (1/0), for each ligand/receptor, across the compendium of cell types indicating with a 1 if the ligand/receptor is expected to be found on a cell-type based on prior evidence of cell-surface protein expression or secretion (0 otherwise). This knowledge may be extracted from Appendix II-IV of Janeway's Immunobiology 9th Edition Textbook [36, the contents of which are incorporated herein by reference] and systematically organized in a table where row indicates a specific ligand/receptor of interest and a column indicates a specific cell type of interest. The catalog may also record ligands/receptors whose expected cell-type specific distribution is less precisely defined.

For instance, certain cytokines/chemokines are reported to be broadly produced by lymphocytes. Hence, without additional evidence, it is reasonable to expect that such ligands/receptors can also be produced in specific contexts by all cell-types that are lymphocytes. This notion is formalized by defining a functional equivalence class for each cell-type. For instance, the functional equivalence class for B cells is defined as: {lymphocytes, lymphoid cells, leukocytes, antigen presenting cells, nucleated cells, all cells}. A schema representing such relationships is shown in Table 2 wherein each row defined in columns 2 and 3 points to a specific row in column 1 (which holds the cell type name). Each row in column 3 points to all cell types in the functional equivalence class of a given cell type. Whereas column 2 additionally points to all cell types that precede the given cell type in its developmental lineage.

TABLE 2

| Cell type | Lineage | Functional |
|---|---|---|
| thymocytes | 65, 67 | 19, 78, 27, 42, 43 |
| langerhans cells | 15, 85, 33, 65, 66, 67 | 100, 57, 4, 26, 27, 42, 43 |
| dendritic cells | 15, 85, 33, 65, 66, 67 | 100, 57, 26, 27, 42, 43 |
| b cells | 35, 65, 67 | 100, 57, 19, 78, 27, 42, 43 |
| intestine cells | | 42, 43 |
| smooth muscle cells | | 42, 43 |
| endothelial cells | | 42, 43 |
| t cells | 36, 2, 65, 67 | 19, 78, 27, 42, 43 |
| nk cells | 65, 67 | 19, 78, 27, 42, 43 |
| t helper cells | 36, 2, 65, 67 | 72, 9, 19, 78, 27, 42, 43 |
| treg cells | 36, 2, 65, 67 | 19, 78, 27, 42, 43 |
| ilc3 cells | 65, 67 | 32, 19, 78, 27, 42, 43 |
| nkt cells | 65, 67 | 19, 78, 27, 42, 43 |
| monocytes | 85, 33, 65, 66, 67 | 26, 27, 42, 43 |
| macrophages | 15, 85, 33, 65, 66, 67 | 100, 57, 26, 27, 42, 43 |
| pluripotential hematopoietic cells | 65, 67 | 43 |
| cd8 t cells | 36, 2, 65, 67 | 9, 19, 78, 27, 109, 42, 43 |
| lymphocytes | 65, 67 | 42, 43 |
| pre-b cells | 35, 65, 67 | 19, 78, 27, 42, 43 |
| eosinophils | 85, 33, 65, 66, 67 | 25, 58, 26, 27, 42, 43 |
| basophils | 85, 33, 65, 66, 67 | 25, 58, 26, 27, 42, 43 |
| platelets | 45, 33, 65, 66, 67 | 26, 43 |
| stromal cells | | 42, 43 |
| granulocytes | 85, 33, 65, 66, 67 | 58, 26, 27, 42, 43 |
| myeloid cells | 33, 65, 66, 67 | 42, 43 |
| leukocytes | | 42, 43 |
| myelomonocytic cells | | 26, 27, 42, 43 |
| neutrophils | 85, 33, 65, 66, 67 | 25, 58, 26, 27, 42, 43 |
| memory t cells | 36, 2, 65, 67 | 72, 18, 19, 78, 27, 42, 43 |
| follicular dendritic cells | | 70, 24, 42, 43 |
| ilc cells | 65, 67 | 19, 78, 27, 42, 43 |
| myeloid progenitor cells | | 42, 43 |
| erythrocytes | | 42, 43 |
| early b cells | 65, 67 | 19, 78, 27, 42, 43 |
| early t cells | 2, 65, 67 | 9, 19, 78, 27, 42, 43 |
| germinal center b cells | 35, 65, 67 | 100, 5, 57, 19, 27, 42, 43 |
| plasma cells | 65, 67 | 19, 78, 27, 42, 43 |
| basal epithelial cells | | 46, 42, 43 |
| hematopoietic cells | | 42, 43 |
| naive t cells | 36, 2, 65, 67 | 72, 18, 9, 19, 78, 27, 42, 43 |
| nucleated cells | | |
| all cells | | |
| neuronal cells | | 42, 43 |
| megakaryocytes | 33, 66, 65, 67 | 42, 43 |
| epithelial cells | | 42, 43 |
| osteoclasts | | 42, 43 |
| many adherent cells | | 42, 43 |
| broad distribution of cells | | 42, 43 |
| trophoblasts | | 42, 43 |
| nonhematopoietic cells | | 42, 43 |
| hematopoietic and non-hematopoietic cells | | 42, 43 |
| keratinocytes | | 42, 43 |
| colon cancer cells | | 56, 57, 100, 42, 43, 119 |
| NK cells | 65, 67 | 19, 78, 27, 42, 43 |
| all proliferating cells | | 100, 57, 42, 43 |
| mhc class ii positive cells | | 42, 43 |
| polymorphonuclear leukocytes | 85, 33, 65, 66, 67 | 25, 26, 27, 42, 43 |
| mast cells | 33, 65, 66, 67 | 26, 27, 42, 43 |
| cd34+ prothymocytes (human) | | 42, 43 |
| all human cell lines | | 42, 43 |
| schwann cells | | 42, 43 |
| bone marrow cells | | 42, 43 |
| bone marrow stem cells | | 42, 43 |
| hematopoietic precursor cells | | 42, 43 |
| myeloid progenitors | | 42, 43 |
| stem/progenitor cells | | 42, 43 |
| cns | | 42, 43 |
| astrocytes | | 42, 43 |
| mesenchymal stromal cells | | 24, 42, 43 |
| fibroblasts | | 24, 42, 43 |
| cd41 cells | 36, 2, 65, 67 | 9, 19, 78, 27, 42, 43 |
| bone marrow stromal cells | | 24, 42, 43 |
| thymic epithelial cells | | 46, 42, 43 |
| pancreatic islet cells | | 42, 43 |
| normal transformed epithelial cells | | 100, 57, 42, 43 |
| breast cancer cells | | 100, 56, 57, 42, 43, 119 |
| lymphoid cells | | 42, 43 |
| malignant b cells | 35, 65, 67 | 100, 57, 5, 19, 78, 27 |
| imcd34+ hematopoietic stem cells | 65, 67 | 42, 43 |
| tfh cells | 36, 2, 65, 67 | 11, 72, 9, 19, 78, 27, 42, 43 |
| th17 cells | 36, 2, 65, 67 | 11, 72, 9, 19, 78, 27, 42, 43 |
| th1 cells | 36, 2, 65, 67 | 11, 72, 9, 19, 78, 27, 42, 43 |
| th2 cells | 36, 2, 65, 67 | 11, 72, 9, 19, 78, 27, 42, 43 |
| promyelocytic cells | 33, 65, 66, 67 | 26, 27, 42, 43 |
| gamma delta t cells | 36, 2, 65, 67 | 72, 18, 9, 19, 78, 27, 109, 42, 43 |
| intestinal t cells | 36, 2, 65, 67 | 72, 18, 19, 78, 27, 42, 43 |
| interdigitating dendritic cells | 15, 85, 33, 65, 66, 67 | 100, 57, 26, 27, 42, 43 |
| normal and infected cells | | 100, 57, 42, 43 |
| neuroblastoma cells | | 56, 57, 100, 42, 43 |
| erythroid cells | | 43 |
| nonerythroid cells | | 42, 43 |
| testes | | 42, 43 |
| adipocytes | | 42, 43 |
| pericytes | | 42, 43 |
| podocytes | | 42, 43 |
| osteoblasts | | 42, 43 |
| cd33+ myeloid cells | 33, 65, 66, 67 | 26, 42, 43 |
| mesenchymal stem cells | | 42, 43 |
| antigen-presenting cells | | 42, 43 |
| ilc2 cells | 65, 67 | 32, 19, 78, 27, 42, 43 |
| many different immune cells | 65, 67 | 42, 43 |
| phages | | |
| microglia | 15, 85, 33, 65, 66, 67 | 100, 57, 16, 42, 43 |
| cardiomyocytes | | 42, 43 |
| plasmacytoid dendritic cells | 65, 67 | 100, 57, 27, 42, 43 |
| variety of hematopoietic cells | | 42, 43 |
| memory b cells | 35, 65, 67 | 100, 57, 5, 19, 78, 27, 42, 43 |
| intraepithelial lymphocytes | 36, 2, 65, 67 | 18, 9, 19, 78, 27, 86, 42, 43 |
| hepatocytes | | 42, 43 |
| kupffer cells | | 100, 57, 16, 42, 43 |
| th22 cells | 36, 2, 65, 67 | 11, 72, 9, 19, 78, 27, 42, 43 |
| high endothelial venules | | 8, 42, 43 |
| lung epithelial cells | | 46, 42, 43 |
| skin epithelial cells | | 46, 42, 43 |
| ebv transformed cells | | 76, 57, 100, 42, 43 |
| chondrocytes | | 42, 43 |
| pituitary cells | | 42, 43 |
| cancer cells | | 100, 57, 56, 42, 43 |
| dust cells | | 100, 57, 16, 42, 43 |
| cd8 dendritic cells | 15, 85, 33, 65, 66, 67 | 100, 57, 26, 27, 42, 43 |
| ilc1 cells | 65, 67 | 32, 19, 78, 27, 42, 43 |

### c. Annotation of Functional Effects of Ligand-Receptor Interactions on Participating Cell Types

Certain ligand-receptor interactions between immune cell types may have an activating or inhibitory effect on the cell type expressing the receptor (also known as the target cell type) or in some cases both the ligand and receptor expressing cell types (regarded in literature as costimulatory). Discovery of such interactions resulted in the development of immune checkpoint blockade therapy such as anti-PD1 and anti-CTLA4 which has revolutionized cancer treatment. Literature on all such interactions may be systematically curated, such as from [40]-[49], and classified them into two ontologies as follows:

1. Activating/costimulatory encapsulates interactions with the following functional characteristics reported in literature: increased cytotoxicity, increased cytokine production, increased cell proliferation, increased cell survival, existence of immunoreceptor tyrosine-based activation motifs (ITAMs) in the cytoplasmic tail of the receptor.

2. Inhibitory/checkpoint encapsulates the following functional characteristics reported in literature: decreased cytotoxicity, exhaustion, reduced cytokine production, decreased TCR signaling activity (for T cells), reduced cell proliferation, reduced cell survival, existence of Immunoreceptor tyrosine-based inhibitory motifs (ITIMs) in the cytoplasmic tail of the receptor.

The functional effect of each interaction additionally depends on the binding affinity of the ligand to the target receptor. In the database, interactions with conflicting effects reported on target cell types or for cases where the effect of the interaction depends on other factors may be left unannotated. In addition to activating/inhibitory interactions, the catalog may also comprise annotations other interactions based on prior knowledge from Janeway's immunobiology 9th Edition Textbook [36].

1. Pro-inflammatory interactions involving inflammation mediator cytokines such Interferon Gamma, TNF-alpha, IL 1, IL12 and IL18

2. Chemotaxis: cytokine/chemokine interactions involved in cell chemotaxis in regular or inflammatory conditions (responsible for lymphocyte infiltration).

### d. LIRICS STEP 1: Querying All Plausible Ligand Receptor Interactions Between any Two Cell-Types Based on Prior Knowledge

The method may comprise querying all ligand receptor interactions that could potentially take place between two cell types A and B (also referred to as a first cell type and a second cell type). The names of any two cell types whose names match with the names in a data source of cell types, as shown in column 1 of Table 2, may be provided, and then LIRICS may list all ligand-receptor interactions that could potentially take place between cell types A and B. The list may be determined by first finding which ligands/receptors are likely to be expressed/secreted by each cell type (cell type A and B). To determine this, the method may comprise first querying the data source of cell types (which may be Table 2) which may catalog the expected distribution of ligands/receptors on different cell types. The method further comprises listing any additional ligands/receptors that are expected to be found in the functional equivalence class of a cell type (specified in column 3 of Table 2). Given a set of all potential ligands and receptors on each cell-type, the method may comprise returning all known physical protein-protein interactions involving these ligands and receptors, for example as shown in Table 1 herein.

### e. LIRICS STEP 2: Identifying which Plausible Interactions are Likely to Occur (or “active”) in Each Sample Given Deconvolved Gene Expression Data from CODEFACS

Given a queried set of all plausible ligand receptor interactions between cell types (A,B,C, . . . ): {(L₁^{A},R₁^{B}), (L₂^{A},R₂^{C}), . . . , (L₁^{C},R₁^{B}), . . . },one can integrate this prior knowledge with deconvolved expression data from CODEFACS to infer which interactions are likely to occur in each sample as follows:

For any two cell types A and B with a plausible ligand-receptor interaction (Lᵢ^{A},Rᵢ^{B}), a binary indicator may be defined as I_{(L}_{ᵢ}_{^{A}}_{,R}_{ᵢ}_{^{B}}₎∈{0,1}, such that I_{(L}_{ᵢ}_{^{A}}_{,R}_{ᵢ}_{^{B}}₎=1 if a physical interaction between (Lᵢ^{A},Rᵢ^{B}) is likely to take place in a sample, and has the value 0 otherwise. An interaction is considered likely to take place (synonym: “active”) in a sample if the ligand Lᵢ^{A} is overexpressed in cell type A and receptor Rᵢ^{B} is over expressed in cell type B, in that sample. To determine if ligand Lᵢ^{A} and receptor Rᵢ^{B} are over-expressed in cell types A and B in a given sample, we use the median deconvolved expression of the ligand Lᵢ^{A} in cell type A and likewise the median deconvolved expression of receptor Rᵢ^{B} in cell type B as controls. Ligands such as cytokines and chemokines, can be secreted by cells and hence, are not surface bound. However, the levels of secreted cytokines/chemokines by a cell type may be expected to be proportional to their cell-type-specific gene expression. Furthermore, certain ligands/receptors can be encoded by multiple genes (each gene being part of a specific subunit in the protein complex). For such ligands/receptors to be expressed, all genes required to build the ligand or receptor need to be expressed. Hence, their expression in a cell type may be modeled as the minimum of the expression of individual genes constituting the ligand or receptor.

This approach has two key advantages, besides being biologically intuitive. First, the binary indicator is expected to be robust to noise in gene expression despite the varying levels of confidence in the predicted cell-type-specific gene expression from different datasets. This follows from the statistical properties of median-based filters in signal processing [50]. Second, it enables comparison of individual profiles independent of the dataset source due to their shared biological representation if the datasets being compared have expression measurements of the same genes. Thus, one can seamlessly pool multiple datasets together, augment sample size and increase statistical power.

### f. Downstream Enrichment Analysis and Visualization

Given this binarized representation, the method may comprise performing a Fisher's exact test to assess if any specific cell-cell interaction is more likely to occur in samples with a specific phenotype compared to a control group. This may be quantified by computing the enrichment score, expressed as an odds ratio of the interaction in each phenotype of interest. A score around 1 indicates a neutral trend, a score>1 indicates enrichment of the interaction in the phenotype of interest and a score close to 0 indicates enrichment in the control group. Furthermore, the associated p-values of each test can be inspected post multiple hypothesis testing correction to identify any significant trends in the data. One can also plot the most significant trends occurring in a network where each edge represents a ligand-receptor interaction between two cell types and the thickness of the edge is proportional to the enrichment score of the interaction in a phenotype of interest. The circlize package in R may be used to make these plots [51].

### 3. Systems

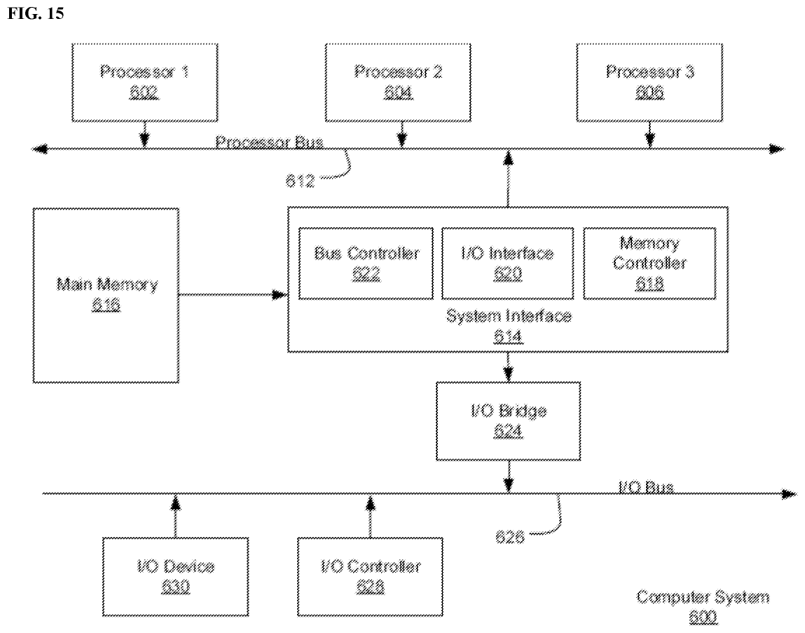

FIG. 15 is a block diagram illustrating an example of a computing device or computer system 600 which may be used in implementing the embodiments of the components of the methods disclosed above. For example, the computing system 600 of FIG. 15 may perform one or more of the operations discussed above. The computer system (system) includes one or more processors 602-606. Processors 602-606 may include one or more internal levels of cache (not shown) and a bus controller or bus interface unit to direct interaction with the processor bus 612. Processor bus 612, also known as the host bus or the front side bus, may be used to couple the processors 602-606 with the system interface 614. System interface 614 may be connected to the processor bus 612 to interface other components of the system 600 with the processor bus 612. For example, system interface 614 may include a memory controller 614 for interfacing a main memory 616 with the processor bus 612. The main memory 616 typically includes one or more memory cards and a control circuit (not shown). System interface 614 may also include an input/output (I/O) interface 620 to interface one or more I/O bridges or I/O devices with the processor bus 612. One or more I/O controllers and/or I/O devices may be connected with the I/O bus 626, such as I/O controller 628 and I/O device 640, as illustrated.

I/O device 640 may also include an input device (not shown), such as an alphanumeric input device, including alphanumeric and other keys for communicating information and/or command selections to the processors 602-606. Another type of user input device includes cursor control, such as a mouse, a trackball, or cursor direction keys for communicating direction information and command selections to the processors 602-606 and for controlling cursor movement on the display device.

System 600 may include a dynamic storage device, referred to as main memory 616, or a random access memory (RAM) or other computer-readable devices coupled to the processor bus 612 for storing information and instructions to be executed by the processors 602-606. Main memory 616 also may be used for storing temporary variables or other intermediate information during execution of instructions by the processors 602-606. System 600 may include a read only memory (ROM) and/or other static storage device coupled to the processor bus 612 for storing static information and instructions for the processors 602-606. The system set forth in FIG. 6 is but one possible example of a computer system that may employ or be configured in accordance with aspects of the present disclosure.

According to one embodiment, the above techniques may be performed by computer system 600 in response to processor 604 executing one or more sequences of one or more instructions contained in main memory 616. These instructions may be read into main memory 616 from another machine-readable medium, such as a storage device. Execution of the sequences of instructions contained in main memory 616 may cause processors 602-606 to perform the process steps described herein. In alternative embodiments, circuitry may be used in place of or in combination with the software instructions. Thus, embodiments of the present disclosure may include both hardware and software components.

A machine readable medium includes any mechanism for storing or transmitting information in a form (e.g., software, processing application) readable by a machine (e.g., a computer). Such media may take the form of, but is not limited to, non-volatile media and volatile media. Non-volatile media includes optical or magnetic disks. Volatile media includes dynamic memory, such as main memory 616. Common forms of machine-readable medium may include, but is not limited to, magnetic storage medium (e.g., floppy diskette); optical storage medium (e.g., CD-ROM); magneto-optical storage medium; read only memory (ROM); random access memory (RAM); erasable programmable memory (e.g., EPROM and EEPROM); flash memory; or other types of medium suitable for storing electronic instructions.

Embodiments of the present disclosure include various steps, which are described in this specification. The steps may be performed by hardware components or may be embodied in machine-executable instructions, which may be used to cause a general-purpose or special-purpose processor programmed with the instructions to perform the steps. Alternatively, the steps may be performed by a combination of hardware, software and/or firmware.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations together with all equivalents thereof.

### Example 1

### Validation of CODEFACS

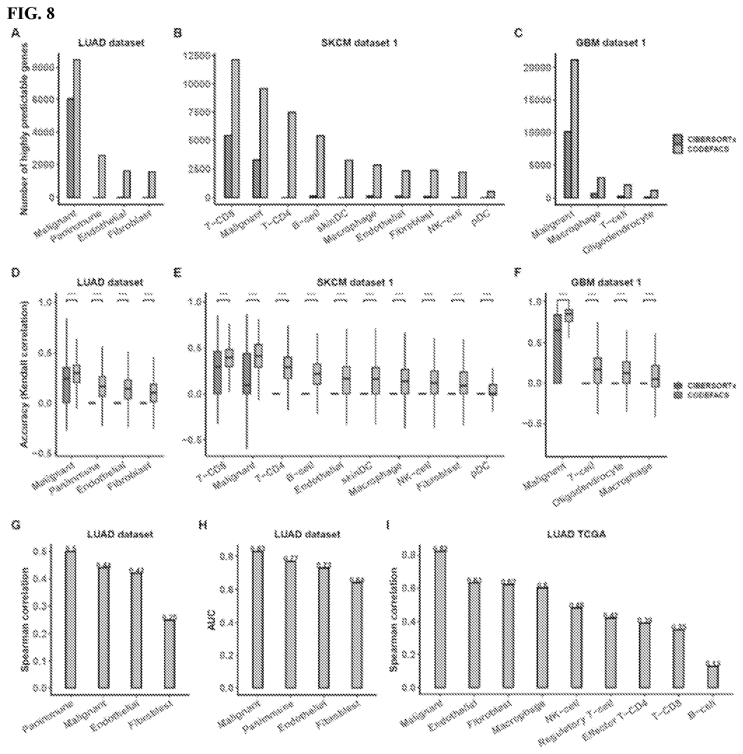

To test the performance of CODEFACS, we generated 15 benchmark datasets as described herein by merging publicly available single cell RNA-seq [8], [9] and FACS sorted purified RNA-seq [10]. Thereafter, we applied CODEFACS to deconvolve these generated bulk datasets and define the accuracy of our predictions, by computing the Kendall correlation between the predicted and ground truth expression in each cell type across individual samples (the Kendall correlation provides a less inflated measure of accuracy by accounting for ties in the data). In the main text, we show the results obtained in the three benchmark datasets that are generated form a FACS-sorted lung cancer dataset, a single cell melanoma RNA-seq dataset and a single cell glioblastoma RNA-seq respectively. We show that CODEFACS can predict the cell-type-specific expression of more genes than CIBERSORTx (with Kendal's correlation≥0.3) (FIG. 8A-C), and its predictions are overall more accurate (FIG. 8D-F). The results for all the remaining 12 benchmark datasets, created via different simulated data generation procedures, also show the superiority of CODEFACS. Overall, it was observed that the more abundant the cell type is, the better CODEFACS can predict its cell-type-specific gene expression.

To test the accuracy of CODEFACS confidence estimations, we quantified how well so the confidence scores it returns align with the Kendal scores for each (gene, cell-type) pair. We quantified this using two metrics: Spearman correlation and a classification AUC (Area Under the ROC Curve). Across all the benchmark datasets analyzed, the Spearman correlation between confidence scores of genes in each cell-type and their corresponding Kendal scores (quantifying the true prediction accuracy) is strong and positive (FIG. 8G depicts the results for the FACS sorted lung cancer benchmark dataset); To perform a classification based quantification, we grouped the genes in each cell-type into two classes based on the correlation between their predicted and actual expression, informative (prediction accuracy≥0.1) and uninformative (prediction accuracy<0.1). We then tested whether the confidence scores could be used to classify genes into these two classes for each cell-type. We found that the confidence score could effectively filter out uninformative predictions (FIG. 8H depicts the results for the FACS sorted lung cancer benchmark dataset).

To further validate CODEFACS we applied it to deconvolve bulk expression data from 21 cancer types (7824 RNA-seq samples) in TCGA. To infer the cellular abundance of each cell type in each sample in the initial step of CODEFACS, we made use of matched bulk methylation data available for these samples and methylation-based reference signature profiles of distinct cell types. These include 11 cell type signatures (macrophages/dendritic cells-CD14+, B cells-CD19+, CD4+ T cells, CD8+ T cells, T reg cells, NK cells-CD56+, endothelial cells, fibroblasts, neutrophils, basophils, eosinophils and tissue-specific tumor cells) obtained from MethylCIBERSORT [11] (Methods). Reassuringly, we found strong Spearman correlations between the resulting predicted cancer cell fraction and tumor purity estimates derived from matched mutation and copy number data on the same samples (based on ABSOLUTE) across 10 cancer types (Spearman correlation: min=0.72, max=0.88, avg=0.8). This testifies that methylation-based cell fraction estimates indeed form a reliable basis for running CODEFACS to deconvolve TCGA samples.

To test if CODEFACS can then recover the expected cell-type-specific gene expression signature of different cell types in a given cancer type, we computed the Spearman correlation between the mean deconvolved gene expression of the top confidently deconvolved genes in a given cell type (confidence score≥0.95) and the mean expression of these genes, which we derived from completely independent single cell expression data of the same cancer type (Methods), finding that they are substantially correlated (FIG. 8I depicts results for the TCGA-LUAD dataset). The concordance level is higher for cell types that are highly abundant (e.g. tumor cells and fibroblasts) and decreases for less abundant cell types. Additionally, we observed that tumor cells have the largest fraction of genes whose expression is predicted with high confidence, with the highest in thyroid cancer (THCA, ˜74.1% of all genes). Furthermore, 12 KEGG pathways are significantly enriched (adjusted p-value<0.01) with highly confident genes in the tumor cells (confidence score≥0.95) across 21 cancer types. Those pathways are mostly relevant to gene regulation, cell cycle and mismatch repair.

### Tumors With DNA Mismatch Repair Deficiency have Heightened T Cell Co-Stimulation that is Independent of their Tumor Mutation Burden Levels

In normal cells, DNA is constantly repaired in response to DNA damage or DNA replication errors [12]. However, defects in specific DNA repair pathways in cancer cells may result in the accumulation of many somatic mutations resulting in hypermutated tumors (TMB>10 mutation/Mb) [13]-[15]. One of the sources of hypermutability is a mismatch repair deficiency (MMRD), which leads to the accumulation of insertions and deletion mutations in microsatellite regions of the genome due to uncorrected DNA replication polymerase slippage events. This is known as microsatellite instability (MSI) [16], [17]. Solid tumors with high MSI levels were shown to be sensitive to immune checkpoint blockade (ICB) therapy irrespective of tumor type, leading the FDA to approve MSI as the first cancer type agnostic biomarker for patients receiving anti-PD1 treatment [18]. The reason behind this general sensitivity to anti-PD1 treatment is not completely understood. Prior work has led to the prevailing hypothesis that elevated tumor mutation burden in mismatch repair deficient tumors leads to more neoantigens, and thus is more likely to activate a host immune response against tumor cells [16], [19]-[21]. However, not all tumors with elevated tumor mutation burden have similar response rates to anti-PD1 [22], [23], and recent studies have revealed that T cells recognize only a few neoantigens per tumor [24]-[27].

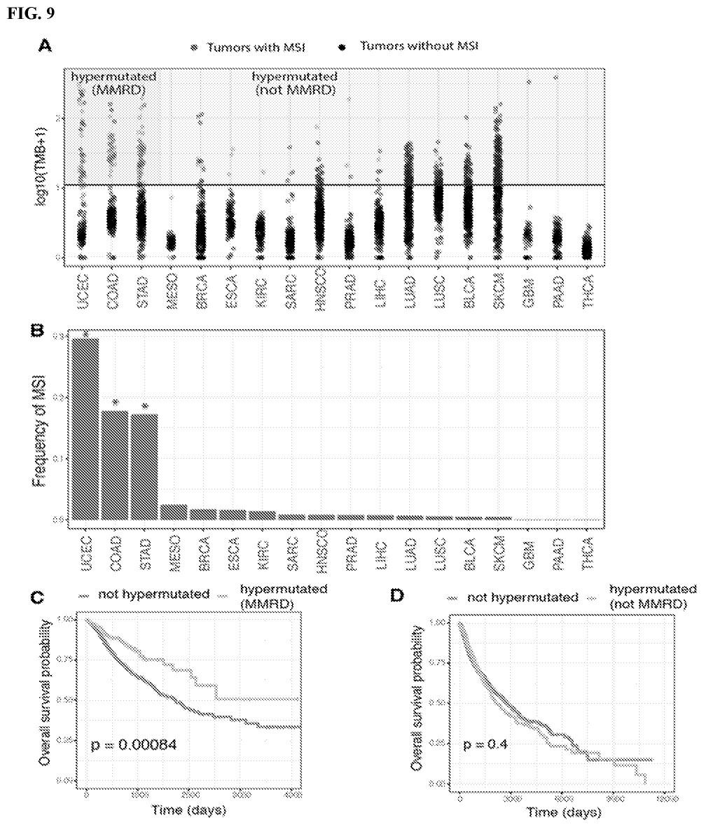

More generally, when looking at TCGA mutation burden, MSI and survival data (FIG. 9A, B, borrowed from [28], [29]), we see a strong association between hypermutability and survival of patients in tumor types with a frequent underlying mismatch repair deficiency, (FIG. 9C. log-rank test p-value=0.00084), in contrast to other tumor types (FIG. 9D, log-rank test p-value=0.4). These survival differences can be partially explained by the mutual exclusivity of microsatellite instability and chromosomal instability, which has been previously linked with tumor-immune evasion and a worse prognosis [28][30].

Taken together, these findings motivated us to study cellular immune crosstalk in the tumor microenvironment of mismatch repair deficient tumors to gain additional cell-type-specific insights into their sensitivity to anti-PD1.

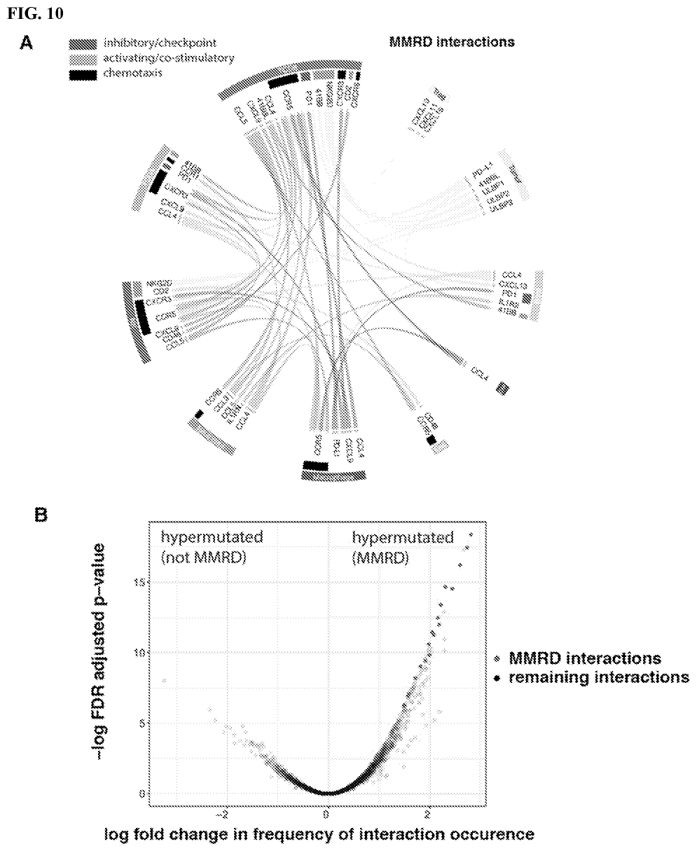

We applied CODEFACS to deconvolve the bulk gene expression of all solid tumors from TCGA and integrated their predicted cell-type-specific gene expression levels with LIRICS to identify cell-cell interactions that are differentially active between microsatellite instable tumors (highlighted as darker grey dots in FIG. 9A) and microsatellite stable tumors (highlighted as black dots in FIG. 9A). The top 50 interactions from this differential analysis (ordered by FDR adjusted p-value) are shown in a network in FIG. 10A. These interactions are indeed frequently active in mismatch repair deficient tumors in distinct tumor types confirming that the differential analysis was not confounded by tumor type specific differences. Furthermore, we see that they are more frequently active in hypermutated tumors with DNA mismatch repair deficiency compared to other hypermutated tumors, (FIG. 10B), testifying to their MSI specificity. The top 50 MSI-specific interactions include the PDL1-PD1 checkpoint interaction between tumor cells/macrophages and CD8+ T-cells, immune cell activating/co-stimulatory interactions such as the 41BBL-41BB interaction between B and T cells, ULBP1/2/3-KLRK1 between tumor cells and CD8+ T cells/NK cells, CD48-CD2 between NK cells and T cells and interactions involved in lymphocyte trafficking, such as the CXCL9-CXCR3 chemokine interactions between macrophages and NK/T cells.

This shared heightened immune cellular crosstalk identified above in the TME of high MSI tumors suggests that tumor infiltrating CD8+ T cells can be robustly activated by co-stimulatory signals in tumors independent of overall tumor mutation burden, only to be kept in balance by the PD1-PDL1 checkpoint interaction between CD8+ T cells and macrophages/tumor cells. When this interaction is blocked by anti-PD1 treatment, it can lead to favorable response of patients to immune checkpoint blockade therapy. This in turn raises the possibility that the presence of these T cell co-stimulation signals in the TME of tumors may serve as a biomarker for their response to anti-PD1 treatment irrespective of either their tumor mutation burden or MSI status. If further verified experimentally, it reinforces the potential benefits of therapies aimed at enhancing these co-stimulating interactions.

### Machine Learning Guided Discovery of Cell-Type-Specific Ligand-Receptor Interactions that Predict Patient Response to ICB Therapy

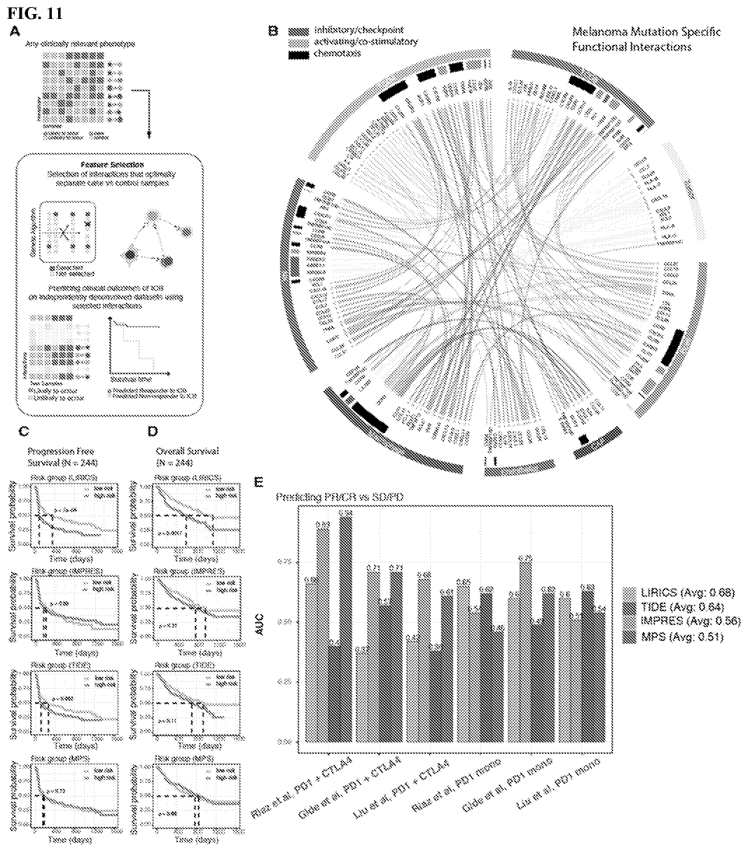

Since some mutations during tumor evolution can be immunogenic, we hypothesized that one could discover cell-type-specific ligand-receptor interactions predictive of response to ICB therapy by a joint analysis of mutation and deconvolved expression data. We focus on melanoma, currently the tumor type best responding to ICB, where we have both many independently generated bulk expression datasets of patient's receiving anti-PD1 treatment and single-cell derived cell-type-specific signatures, which serve as priors for the deconvolution of these bulk datasets. Starting from the TCGA-SKCM dataset as our training set, we employed a genetic algorithm to find cell-type specific ligand-receptor interactions, whose activation state optimally separates hypermutated melanoma tumors from non-hypermutated tumors (This way, we have a sufficient sample size of case vs control samples for training. See FIG. 11A). We term the interactions identified melanoma mutation specific functional interactions (MSFI, the network of these interactions is displayed in FIG. 11B). We additionally applied CODEFACS to deconvolve the bulk expression data of pre-treatment samples from the three largest independently generated melanoma datasets where patients received anti-PD1 treatment (either monotherapy or in combination with anti-CTLA4; Methods) [31]-[33]. Without any additional training, we then applied LIRICS to quantify the number of MSFI interactions that are active in each test sample, which we denote as its MSFI score. Remarkably, we find that the MSFI score of each sample can robustly stratify patients into those that are likely to respond to ICB vs those that are unlikely to respond (FIG. 11C, progression free survival log rank test p-value: 0.0007, FIG. 11D, overall survival log rank test p-value: 0.001), improving over recent bulk gene expression based predictors for melanoma [34], [35], [36]. In addition, we also plot the area under the curve (AUC) of each score—MSFI, IMPRES [34], TIDE [35] and melanocytic plasticity signature (MPS) scores [36]—in predicting partial/complete responders vs stable/progressive disease patients (FIG. 11E) in these datasets. On average, the MSFI score achieves an AUC of 0.68, followed by TIDE with an average AUC of 0.64, IMPRES with an average AUC of 0.56 and MPS with an average AUC of 0.51. It is however possible that the performance of some of these bulk gene expression-based predictors might have been affected by the choice of RNA-Seq expression quantification method used for preprocessing the raw data, which we discuss further in the limitations of this study.

We additionally asked what is the predictive power of other scores built around the MSFI network? Specifically, we asked what is the predictive power if we (1) only count the number of ligands that are over-expressed in designated cell-types in a given sample (without considering the receptor expression) and vice-versa, or (2) count the number of active interactions with swapped cell-type placements of the ligand and receptor in a given sample, and finally (3) count the number of active interactions upon random shuffling of interaction activation status across samples? The average AUC of the different scores across the different datasets in predicting patient response is 0.65, 0.62 and 0.51, respectively. These results further support the clinical relevance of the MSFI network, which was never trained on any ICB-treated datasets.

Examining the MSFI cell-type-specific ligand-receptor interactions leads to interesting insights. First, we see that the therapeutically relevant PDL1-PD1 checkpoint interaction is selected, but interestingly, it is composed of PDL1 being over-expressed on macrophages and PD1 being over-expressed on CD8+ T cells. Second, we find an over-representation of cell-type-specific co-stimulatory/immune cell activating interactions known from prior immunological literature (hypergeometric test p-value: 1.3E−8) [37][47]. Thirdly, the MSFI network includes many cytokine/chemokine interactions involved in pro-inflammatory response and the trafficking of NK, T and B cells to the tumor site (responsible for better lymphocyte infiltration into the tumor mass). Overall, these results suggest that a pro-inflammatory MSFI-rich TME coupled with appropriate co-stimulatory cues mobilizes tumor infiltrating lymphocytes to generate an effective host immune response upon immune checkpoint blockade in melanoma.

### Discussion

This study presents a computational tool, CODEFACS and a pipeline, LIRICS, that enables an (averaged) ‘virtual single cell’ characterization of the TME from bulk tumor expression data. Applying these tools, we identify cell type specific ligand-receptors interactions that are active and functionally important within individual tumor microenvironments, in modifying patients survival and response to ICB: Applying CODEFACS to 7824 tumors from TCGA, we estimate the cell-type-specific gene expression profiles of each individual tumor sample, for the first time. We provide this information as a publicly available data resource for analysis of the TCGA at a cell-type-specific resolution. Integrating these data with LIRICS, we systematically characterized the immune cellular crosstalk of the tumor microenvironments of different tumor types. We identified a shared core of intercellular TME interactions in DNA mismatch repair deficient tumors, which are associated with improved patient survival and high sensitivity to immune checkpoint blockade therapy. One potentially interesting implication of these findings is that immunomodulators enhancing T-cell co-stimulation (e.g. via 41BB) might improve patients' response to ICB irrespective of their tumor mutation burden. Finally, focusing on melanoma, we show that one can bootstrap on the deconvolved data to discover cell-cell interactions within the melanoma TME that successfully predict patients' response to immune checkpoint blockade.

While we have provided a toolkit to discover clinically relevant cell-cell interactions from bulk tumor expression, there are limitations that should be noted and potentially further improved upon in the future. First, in this study, we relied on Kalisto, a relatively recent and rapid expression quantification method to quickly pre-process all bulk RNA-Seq datasets with publicly available raw read data. Several recent publications have reported discrepancies between alignment-based and alignment-free RNA-seq expression quantification methods for relatively short length or moderate to lowly-expressed genes [48]-[52]. Some of these genes might be biologically relevant and this can consequently affect reproducibility of findings. Hence, whenever possible, we recommend that all bulk RNA-seq datasets are uniformly pre-processed using one RNA-seq quantification method before the application of our tools (See Methods), and any clinically relevant findings are further followed up.

Second, regarding limitations of CODEFACS itself (1) it requires prior information about the cell type composition of the input tumors, or alternatively, knowledge of the pertaining cell-types' gene expression or methylation signatures that can be used to infer their abundances, and its accuracy depends on the accuracy of the latter. (2) its prediction power is limited to subsets of the whole exome genes, and its performance deteriorates for lowly-abundant cell types. The confidence scores provided partially alleviate this limitation, allowing the user to rank genes in each cell-type by the quality of predictions of the expression of each gene in a given cell-type. Third, regarding LIRICS, it currently does not consider the spatial localization of cells in the TME and the inclusion of the latter with the advent of forthcoming spatial transcriptomics data is likely to lead to considerably more informative interaction inference approaches.

Although this work focuses on studying the tumor microenvironment, the tools presented here can be applied to discover important cell-cell interactions in noncancerous tissues under a variety of normal and disease states. An interesting application that we envision is the characterization of clinically relevant intercellular interactions occurring at the maternal-fetal interface using corresponding bulk gene expression data and pregnancy outcome information, whose elucidation may help treat and mitigate preeclampsia and other pregnancy related complications. One can also use our tools to study bulk gene expression data from pre-malignant tissue samples and compare them against malignant samples to discover TME interactions on the way malignancy. Last but not least, one can use our tools to deconvolve expression data from autoimmune disorders to learn more about the underlying immune interactions. In sum, the computational tools developed and presented in this study offer a novel and cost-effective way to study immune responses at a cell type specific resolution from the widely abundant bulk gene expression in both health and disease, complementing first-line single cell technologies in situations where the latter are less applicable.

### Methods

### Data Collection and Pre-Processing

Single cell RNA-seq datasets: To benchmark the performance of CODEFACS, we first set out to obtain publicly available single cell RNA-seq datasets where both tumor and non-tumor cells were successfully isolated. This search led us to the identification of 9 such single cell RNA-seq datasets from the literature, each from a different cancer type. Collection of additional single cell datasets was frozen after December 2019. Details of the single cell datasets that we curated for this study in the following table.

TABLE 3

| | | | Number of |
|---|---|---|---|
| Dataset | Cancer | Sequencing | patients |
| Reference | type | Platform | studied |
| GSE115978 | Melanoma | SmartSeq2 | 32 |
| GSE131928 | GBM | SmartSeq2 | 28 |
| GSE103322 | HNSCC | SmartSeq2 | 18 |
| CRA001160 | PDAC | 10x | 35 |
| GSE125449 | LIHC | SmartSeq2 | 12 |
| GSE81861 | CRC | SmartSeq2 | 11 |
| E-MTAB-6149 | LEAD | 10x | 3 |
| E-MTAB-6149 | LUSC | 10x | 2 |
| GSE118389 | TNBC | SmartSeq2 | 6 |

For each dataset sequenced on the SmartSeq2 platform, the log normalized transcript counts for each gene in each sequenced cell were made publicly available by the original authors. For the application of deconvolution, these counts were transformed back to the Transcripts Per Million (TPM) scale. For datasets sequenced on the 10× platform, UMI counts for each gene were made publicly available and were scaled by the library size of each cell and multiplied by a factor of 1 million to get expression values in TPM scale.

Bulk RNA-seq datasets: Gene expression and matching bulk tumor methylation data from fresh frozen tumor biopsies in TCGA were downloaded from (http://xena.ucsc.edu/) [53]. In addition, publicly available bulk expression data from formalin fixed paraffin embedded tumor biopsies of melanoma patients receiving immune checkpoint blockade treatment were downloaded from [31]-[33]. All bulk RNA-seq datasets were collected such that they have a sufficiently large sample size to reliably perform complete deconvolution of expression profiles (>4 times the number of cell-types involved) [7]. Collection of datasets was frozen after December 2019. Details on bulk RNA-seq datasets deconvolved in this study can be found in the following table.

TABLE 4

| Cohort | | |
|---|---|---|
| Name | Cancer Type(s) | Cohort description |
| The | UCEC, COAD, | 6972 samples spanning 21 distinct |
| Cancer | STAD, MESO, BRCA, ESCA, KIRC, | cancer types in the TCGA with |
| Genome | SARC, HNSCC, PRAD, LIHC, LUAD, | matched bulk methylation profiles. All |
| Atlas | LUSC, Lung not specified, | samples were biopsied pre-treatment. |
| | BLCA, SKCM, GBM, PAAD, | |
| | THCA, AML, DLBC | |
| Riaz et al, | Melanoma | 109 samples from 73 patients, 51 |
| Cell, 2017 | | patients had pre-treatment samples (25 |
| | | anti-PD1 monotherapy, 26 previously |
| | | progressed on anti-CTLA4) |
| Gide | Melanoma | 91 samples from 75 patients, |
| et al, | | 73 patients had pre-treatment samples |
| Cancer | | (41 anti-PD1 monotherapy, |
| Cell 2019 | | 32 anti-PD1+ anti CTLA4) |
| Liu et al, | Melanoma | 121 samples from 121 patients, 120 |
| Nature | | patients had pre-treatment samples |
| Medicine | | (75 anti-PD1 monotherapy, 45 |
| 2019 | | previously progressed on anti-CTLA4) |

To maintain consistency with the pipeline used for preprocessing TCGA data, bulk expression levels of transcripts in immune checkpoint blockade datasets were re-quantified using Kallisto v0.46.0 [54] with GENCODE v23 hg19 human genome annotation [55] (with the exception of [33] for which raw sequencing data were not available at the time of their publication). Expression at the gene level was estimated from transcript level TPM values using tximport v1.0.3 [56] and GENCODE v23 hg19 annotation. Furthermore, to mitigate technical biases, between-sample scaling factors were estimated using TMM method implemented in edgeR [57] and TPM values in each sample were further rescaled by these scaling factors [58].

Pseudo bulk RNA-seq datasets: To robustly evaluate the performance of CODEFACS, we generated 14 different pseudo-bulk RNA-seq datasets from mixing experiments with single cell data. Each sample in each benchmark dataset has matching cell type specific gene expression profiles derived from averaging single cell RNA-seq profiles of individual cells from the same sample and same cell type. These profiles serve as the ground truth for the evaluation of deconvolution performance. To avoid any circularity in our validations, for each of the single cell datasets involved, single cell data from 4 randomly chosen patients were separated from the rest. These data were used to derive reference gene expression signatures for each cell type. The mixing experiments were then performed on single cell data of the remaining patients that were hidden from the reference signature derivation process. In addition, we simulated technical replicates for each pseudo-bulk sample, wherein we injected noise in the pseudo-bulk expression of a few randomly chosen genes and then renormalized the expression data by the sample library size. This procedure simulates mRNA composition noise that is commonly observed in bulk RNA-seq datasets due to technical differences in sample preparation [64-66]. The datasets and the mixing experiment used to generate the pseudo-bulk samples are listed in the following table.

TABLE 5

| Benchmark | |
|---|---|
| Dataset | |
| name | Description |
| SKCM | 28 pseudo bulk melanoma samples generated by averaging single cell RNASeq |
| dataset 1 | expression profiles (GSE115978) from the same patient |
| SKCM | 28 pseudo bulk melanoma samples generated by averaging imputed single cell |
| dataset 2 | RNASeq expression profiles (GSE115978) from the same patient. Imputation of |
| | single cell RNASeq data was performed using scImpute v0.0.9 |
| SKCM | First noisy technical replicate of SKCM dataset 2 generated by injecting noise in |
| dataset 3 | each of the 28 mixes |
| SKCM | Second noisy technical replicate of SKCM dataset 2 generated by injecting noise |
| dataset 4 | in each of the 28 mixes |
| SKCM | 100 pseudo bulk melanoma samples generated by sampling single cell RNASeq |
| dataset 5 | expression profiles (GSE115978) from different patients in varying cell type |
| | specific proportions and averaging them. |
| SKCM | First noisy technical replicate of SKCM dataset 5 generated by injecting noise in |
| dataset 6 | each of the 100 mixes |
| SKCM | Second noisy technical replicate of SKCM dataset 7 generated by injecting noise |
| dataset 7 | in each of the 100 mixes |
| GBM | 24 pseudo bulk GBM samples generated by averaging single cell RNASeq |
| dataset 1 | expression profiles (GSE131928) from the same patient |
| GBM | 24 pseudo bulk GBM samples generated by averaging imputed single cell |
| dataset 2 | RNASeq expression profiles (GSE131928) from the same patient. Imputation of |
| | single cell RNASeq data was performed using scImpute v0.0.9 |
| GBM | First noisy technical replicate of GBM dataset 2 generated by injecting noise in |
| dataset 3 | each of the 24 mixes |
| GBM | Second noisy technical replicate of GBM dataset 2 generated by injecting noise |
| dataset 4 | in each of the 24 mixes |
| GBM | 100 pseudo bulk GBM samples generated by sampling single cell RNASeq |
| dataset 5 | expression profiles (GSE131928) from different patients in varying cell type |
| | specific proportions and averaging them. |
| GBM | First noisy technical replicate of GBM dataset 5 generated by injecting noise in |
| dataset 6 | each of the 100 mixes |
| GBM | Second noisy technical replicate of GBM dataset 5 generated by injecting noise |
| dataset 7 | in each of the 100 mixes |

Procedure for injecting noise into expression profiles of bulk mixtures in order to simulate batch effects:
Step 1: For each sample: pseudo bulk expression values of a randomly chosen subset of genes (500 genes for first technical replicate, and 3000 genes for second) were perturbed randomly.
Step 2: For each sample: the expression values were renormalized to sum up to 1 million transcripts

In addition, we obtained a FACS sorted lung cancer dataset which include purified RNA-seq for four cell types [10] and generated a pseudo bulk correspondingly. In total, 15 benchmark datasets were generated.

### Deconvolving Immune Checkpoint blockade (ICB) and TCGA Bulk RNA-Seq Datasets using CODEFACS

For the application of CODEFACS, molecular profiles of signature genes of each cell type of interest are needed to estimate the relative cell fractions in the bulk. We used single cell expression derived signatures as priors to deconvolve the melanoma ICB datasets. To derive these signatures from single cell data, we first start out by obtaining the class labels of each cell type of interest. These data are publicly available for each single cell dataset we collected. Hence, we primarily use these labels in our study (unless further refinement of labels into specific cell subtypes of interest is needed for a specific application). With a collection of single cell expression profiles and matching cell type labels as input, we used CIBERSORT online tool to derive a cell-type-specific signature matrix. Thereafter, we applied CODEFACS to ICB datasets with default parameters settings and batch correction requirement specified. For TCGA deconvolution, we first estimated cell fractions based on bulk methylation and then applied CODEFACS to corresponding bulk gene expression for the 21 cancer types which have both types of data available. We chose methylation signatures over expression-based signatures for TCGA analysis for two reasons. First, single cell expression data with consistent cell types across 21 cancer types are not available. Second, DNA methylation-based signatures are considered to be more stable marks of cellular identity compared to dynamic RNA expression derived signatures [59]. The methylation-based cell type signatures (see following table) were obtained from MethylCIBERSORT [11]. We applied CODEFACS to TCGA datasets with default parameters settings and without batch correction requirement specified. The details of CODEFACS algorithm and parameter settings are described herein.

TABLE 6
Non-cancer cell types with
methylation signatures
Endothelial
Fibrolast
CD14+ (Monocyte/Macrophages/Dendritic cells)
CD19+ (B cells)
CD56+ (NK cells)
CD4+ (T cells)
CD8+ (T cells)
Treg (T cells)
Eos (Eosinophils)
Neu (Neutrophils)

| | Matched |
|---|---|
| Cancer cell types with | cancer type |
| methylation signatures | (TCGA) |
| endometrium | TCGA-UCEC |
| large_intestine | TCGA-COAD |
| stomach | TCGA-STAD |
| mesothelioma | TCGA-MESO |
| breast | TCGA-BRCA |
| oesophagus | TCGA-ESCA |
| kidney | TCGA-KIRC |
| sarcoma | TCGA-SARC |
| head_and_neck | TCGA-HNSC |
| prostate | TCGA-PRAD |
| liver | TCGA-LIHC |
| lung_NSCLC_adenocarcinoma | TCGA-LUAD |
| lung_NSCLC_squamous_cell_carcinoma | TCGA-LUSC |
| bladder | TCGA-BLCA |
| skin | TCGA-SKCM |
| glioma | TCGA-GBM |
| pancreas | TCGA-PAAD |
| thyroid | TCGA-THCA |
| acute_ myeloid_ eukaemia | TCGA-AML |
| B_cell_lymphoma | TCGA-DLBC |

### Machine Learning for Discovery of Cell-Cell Interactions Predictive of Clinical Responses to Immune Checkpoint Blockade

We use a genetic algorithm designed to select optimal features for a prediction task given some user-defined fitness function [60]. In this setting, the features are ligand-receptor interactions between cell-types, the prediction task is predicting response to ICB treatment and the fitness function is defined as the accuracy of predicting a user defined phenotype based on the total number of interactions from those selected occurring in a given sample; accuracy is quantified by the AUC.

The algorithm starts out by randomly generating sets of features. This is defined as the seed population. These sets iteratively evolve via the phenomenon of natural selection enforced by the user defined fitness function. Specifically, for each subsequent iteration, features from the best performing sets, as determined by the user defined fitness function, in the current iteration are mixed at random followed by random new feature additions or dropouts (referred to as mutations) to build a new generation of feature sets and the process repeats. Eventually, after a number of iterations, which we set to 100, the fitness function converges to an optimum and the best set of features for the prediction task is returned to the user. Since the fitness function is non-convex and the training process is stochastic, we repeat the training process 500 times and eventually choose frequently selected features over all solutions to reach a solution we suspect is close to the global optimum solution. For our plots, we set the threshold to Frequency>100 times. Results are qualitatively similar for other thresholds. The genetic algorithm was implemented in R using the genalg package [61]

### Example 2

### The CODEFACS Method Exhibits Improvement Over Previous Methods

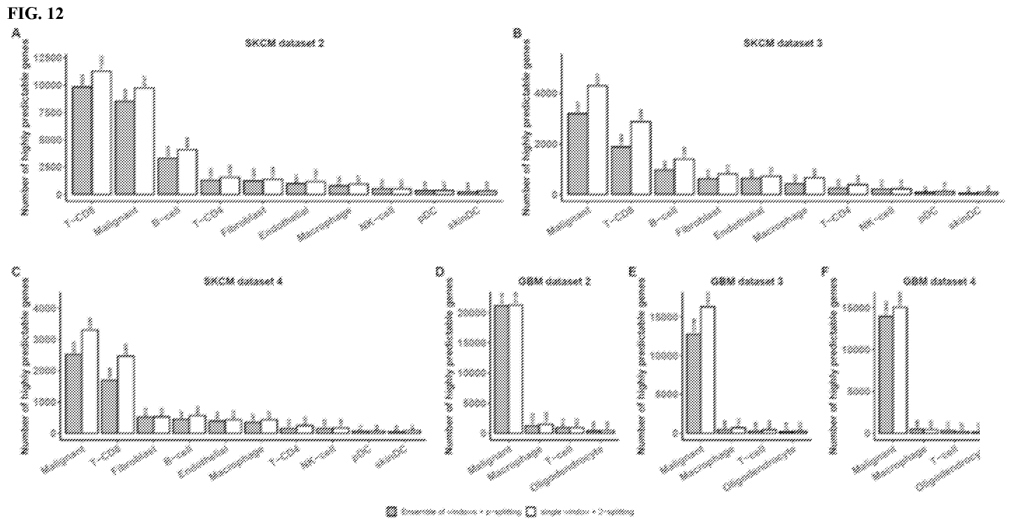

This example demonstrates how the methods disclosed herein are superior to previously known methods. In module 1 of CODEFACS we extended the high-resolution deconvolution approach presented previously in the CIBERSORTx algorithm, by generalizing their two-freedom estimation method to “p-freedom estimation” and modifying their sliding window method to ensemble sliding window deconvolution. By applying to the six benchmark datasets with sufficient sample size (SKCM dataset 2, SKCM dataset 3, SKCM dataset 4, GBM dataset 2, GBM dataset 3, GBM dataset 4; FIG. 12, sample size=100), we showed our recursive splitting (p-freedom estimation) and ensemble of window sizes methods substantially improved the prediction performance (in terms of the number of genes with Kendall scores≥0.3). This improvement is seen especially in abundant cell types and is robust to artificial noise introduced in different pseudo-bulk expression datasets. (FIG. 12B-C and FIG. 12E-F).

### Demonstration of Deconvolution Performance Gains as CODEFACS Proceeds through Each Module

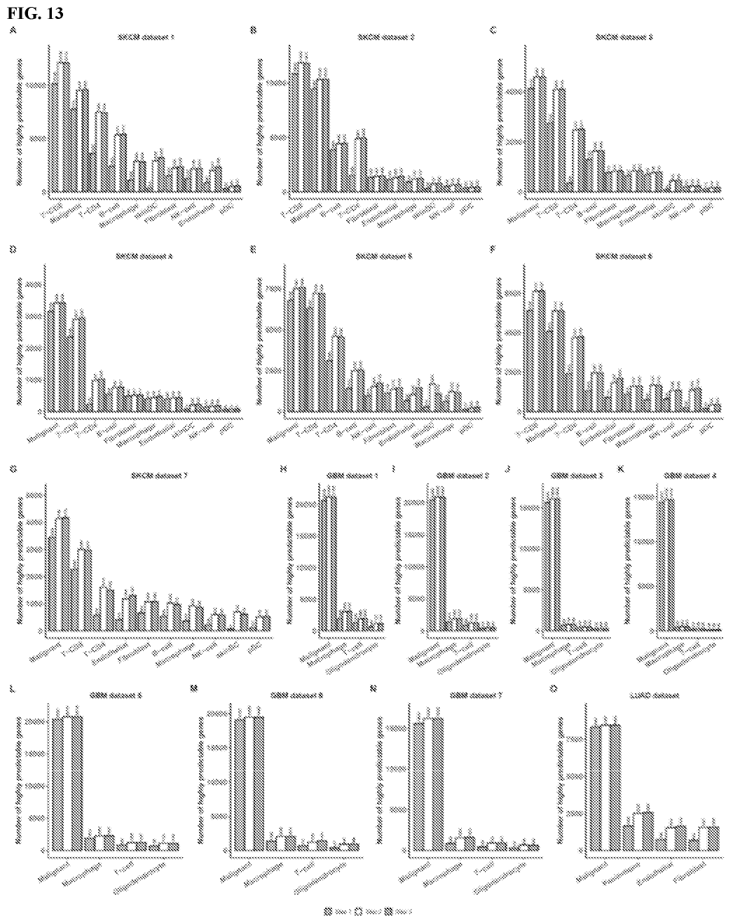

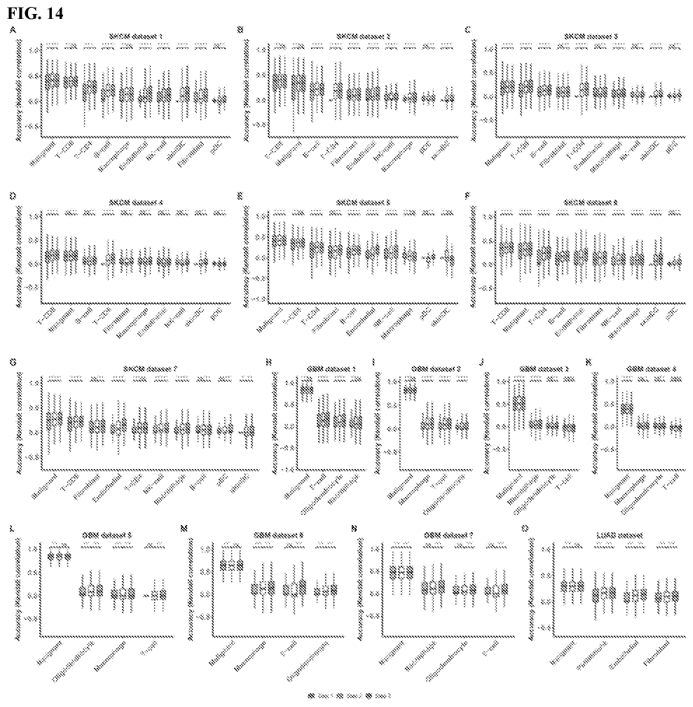

The CODEFACS algorithm relies on a series of heuristics that are expected to greedily predict closer to the ground truth as we sequentially execute each of its three modules. Here we show the performance of this approach in practice using the 15 benchmark datasets we generated by keeping track of the number of genes achieving Kendal scores≥0.3 as the algorithm proceeds through its three modules (FIG. 13) and the Kendal score distribution of high confidence genes at the end of each module (FIG. 14). In general, for each cell type among the 15 benchmark datasets, we showed the overall predictions are improved across the three modules and module 2 achieves the most substantial improvement upon module 1.

### REFERENCES

[1] S. Paget, “the Distribution of Secondary Growths in Cancer of the Breast.,” Lancet, vol. 133, no. 3421, pp. 571-573, 1889, doi: 10.1016/S0140-6736(00)49915-0.

[2] M. J. Smyth, S. F. Ngiow, A. Ribas, and M. W. L. Teng, “Combination cancer immunotherapies tailored to the tumour microenvironment,” Nature Reviews Clinical Oncology, vol. 13, no. 3. pp. 143-158, 2016, doi: 10.1038/nrclinonc.2015.209.

[3] A. Wagner, A. Regev, and N. Yosef, “Revealing the vectors of cellular identity with single-cell genomics,” Nature Biotechnology, vol. 34, no. 11. pp. 1145-1160, 2016, doi: 10.1038/nbt.3711.

[4] Z. Wang et al., “Transcriptome Deconvolution of Heterogeneous Tumor Samples with Immune Infiltration,” iScience, vol. 9, pp. 451-460, November 2018, doi: 10.1016/j.isci.2018.10.028.

[5] G. Quon, S. Haider, A. G. Deshwar, A. Cui, P. C. Boutros, and Q. Morris, “Computational purification of individual tumor gene expression profiles leads to significant improvements in prognostic prediction,” Genome Med., vol. 5, no. 3, 2013, doi: 10.1186/gm433.

[6] N. S. Fox, S. Haider, A. L. Harris, and P. C. Boutros, “Landscape of transcriptomic interactions between breast cancer and its microenvironment,” Nat. Commun., vol. 10, no. 1, pp. 1-13, Dec. 2019, doi: 10.1038/s41467-019-10929-z.

[7] A. M. Newman et al., “Determining cell type abundance and expression from bulk tissues with digital cytometry,” Nat. Biotechnol., vol. 37, no. 7, pp. 773-782, 2019, doi: 10.1038/s41587-019-0114-2.

[8] L. Jerby-Arnon et al., “A Cancer Cell Program Promotes T Cell Exclusion and Resistance to Checkpoint Blockade,” Cell, vol. 175, no. 4, pp. 984-997.e24, 2018, doi: 10.1016/j.cell.2018.09.006.

[9] U. Ghoshdastider et al., “Data-driven inference of crosstalk in the tumor microenvironment,” bioRxiv, p. 835512, January 2019, doi: 10.1101/835512.

[10] A. J. Gentles et al., “A human lung tumor microenvironment interactome identifies clinically relevant cell-type cross-talk,” Genome Biol., vol. 21, no. 1, p. 107, May 2020, doi: 10.1186/s13059-020-02019-x.

[11] A. Chakravarthy et al., “Pan-cancer deconvolution of tumour composition using DNA methylation,” Nat. Commun., vol. 9, no. 1, pp. 1-13, December 2018, doi: 10.1038/s41467-018-05570-1.

[12] J. W. Drake, B. Charlesworth, D. Charlesworth, and J. F. Crow, “Rates of spontaneous mutation,” Genetics, vol. 148, no. 4, pp. 1667-1686, 1998, doi: 10.1007/978-94-011-4385-1 17.

[13] L. A. Loeb, “Mutator Phenotype May Be Required for Multistage Carcinogenesis,” Cancer Res., vol. 51, no. 12, pp. 3075-3079, 1991.

[14] L. B. Alexandrov et al., “Signatures of mutational processes in human cancer,” Nature, vol. 500, no. 7463, pp. 415-421, 2013, doi: 10.1038/nature12477.

[15] D. M. Muzny et al., “Comprehensive molecular characterization of human colon and rectal cancer,” Nature, vol. 487, no. 7407, pp. 330-337, 2012, doi: 10.1038/nature11252.

[16] W. K. Funkhouser et al., “Relevance, Pathogenesis, and testing algorithm for mismatch repair-defective colorectal carcinomas: A report of the association for molecular pathology,” J. Mol. Diagnostics, vol. 14, no. 2, pp. 91-103, 2012, doi: 10.1016/j.jmoldx.2011.11.001.

[17] I. Cortes-Ciriano, S. Lee, W. Y. Park, T. M. Kim, and P. J. Park, “A molecular portrait of microsatellite instability across multiple cancers,” Nat. Commun., vol. 8, 2017, doi: 10.1038/ncomms15180.

[18] M. M. Boyiadzis, J. M. Kirkwood, J. L. Marshall, C. C. Pritchard, N. S. Azad, and J. L. Gulley, “Significance and implications of FDA approval of pembrolizumab for biomarker-defined disease,” Journal for ImmunoTherapy of Cancer, vol. 6, no. 1. 2018, doi: 10.1186/s40425-018-0342-x.

[19] J. Galon et al., “Type, density, and location of immune cells within human colorectal tumors predict clinical outcome,” Science (80-.)., vol. 313, no. 5795, pp. 1960-1964, 2006, doi: 10.1126/science.1129139.

[20] N. J. Llosa et al., “The vigorous immune microenvironment of microsatellite instable colon cancer is balanced by multiple counter-inhibitory checkpoints,” Cancer Discov., vol. 5, no. 1, pp. 43-51, 2015, doi: 10.1158/2159-8290.CD-14-0863.

[21] D. T. Le et al., “PD-1 blockade in tumors with mismatch-repair deficiency,” N. Engl. J. Med., vol. 372, no. 26, pp. 2509-2520, 2015, doi: 10.1056/NEJMoa1500596.

[22] M. Yarchoan, A. Hopkins, and E. M. Jaffee, “Tumor Mutational Burden and Response Rate to PD-1 Inhibition,” N. Engl. J. Med., 2017, doi: 10.1056/nejmc1713444.

[23] M. Imakaev, “Limited evidence of tumour mutational burden as a biomarker of response to immunotherapy,” bioRxiv, p. 2020.09.03.260265, January 2020, doi: 10.1101/2020.09.03.260265.

[24] P. F. Robbins et al., “Mining exomic sequencing data to identify mutated antigens recognized by adoptively transferred tumor-reactive T cells,” Nat. Med., vol. 19, no. 6, pp. 747-752, 2013, doi: 10.1038/nm.3161.

[25] M. Bassani-Sternberg et al., “Direct identification of clinically relevant neoepitopes presented on native human melanoma tissue by mass spectrometry,” Nat. Commun., vol. 7, 2016, doi: 10.1038/ncomms13404.

[26] W. Roh et al., “Integrated molecular analysis of tumor biopsies on sequential CTLA-4 and PD-1 blockade reveals markers of response and resistance,” Sci. Transl. Med., vol. 9, no. 379, 2017, doi: 10.1126/scitranslmed.aah3560.

[27] S. Kalaora et al., “Combined analysis of antigen presentation and T-cell recognition reveals restricted immune responses in melanoma,” Cancer Discov., vol. 8, no. 11, pp. 1366-1375, 2018, doi: 10.1158/2159-8290.CD-17-1418.

[28] A. M. Taylor et al., “Genomic and Functional Approaches to Understanding Cancer Aneuploidy,” Cancer Cell, 2018, doi: 10.1016/j.ccell.2018.03.007.

[29] R. Bonneville et al., “Landscape of Microsatellite Instability Across 39 Cancer Types,” JCO Precis. Oncol., no. 1, pp. 1-15, 2017, doi: 10.1200/po.17.00073.

[30] T. Davoli, H. Uno, E. C. Wooten, and S. J. Elledge, “Tumor aneuploidy correlates with markers of immune evasion and with reduced response to immunotherapy,” Science (80-.)., 2017, doi: 10.1126/science.aaf8399.

[31] N. Riaz et al., “Tumor and Microenvironment Evolution during Immunotherapy with Nivolumab,” Cell, vol. 171, no. 4, pp. 934-949.e15, 2017, doi: 10.1016/j.cell.2017.09.028.

[32] T. N. Gide et al., “Distinct Immune Cell Populations Define Response to Anti-PD-1 Monotherapy and Anti-PD-1/Anti-CTLA-4 Combined Therapy,” Cancer Cell, vol. 35, no. 2, pp. 238-255.e6, 2019, doi: 10.1016/j.ccell.2019.01.003.

[33] D. Liu et al., “Integrative molecular and clinical modeling of clinical outcomes to PD1 blockade in patients with metastatic melanoma,” Nat. Med., vol. 25, no. 12, pp. 1916-1927, 2019, doi: 10.1038/s41591-019-0654-5.

[34] N. Auslander et al., “Robust prediction of response to immune checkpoint blockade therapy in metastatic melanoma,” Nat. Med., vol. 24, no. 10, pp. 1545-1549, 2018, doi: 10.1038/s41591-018-0157-9.

[35] P. Jiang et al., “Signatures of T cell dysfunction and exclusion predict cancer immunotherapy response,” Nat. Med., vol. 24, no. 10, pp. 1550-1558, 2018, doi: 10.1038/s41591-018-0136-1.

[36] E. Pérez-Guijarro et al., “Multimodel preclinical platform predicts clinical response of melanoma to immunotherapy,” Nat. Med., vol. 26, no. 5, pp. 781-791, 2020, doi: 10.1038/s41591-020-0818-3.

[37] D. D. Billadeau and P. J. Leibson, “ITAMs versus striking a balance during cell regulation,” J. Clin. Invest., vol. 109, no. 2, pp. 161-168, 2002, doi: 10.1172/jci14843.

[38] E. Staub, A. Rosenthal, and B. Hinzmann, “Systematic identification of immunoreceptor tyrosine-based inhibitory motifs in the human proteome,” Cell. Signal., vol. 16, no. 4, pp. 435-456, 2004, doi: 10.1016/j.cellsig.2003.08.013.

[39] K. Murphy and C. Weaver, Janeway's Immunobiology. Ninth Edition. 2018.

[40] L. Chen and D. B. Flies, “Molecular mechanisms of T cell co-stimulation and co-inhibition,” Nature Reviews Immunology, vol. 13, no. 4. pp. 227-242, 2013, doi: 10.1038/nri3405.

[41] K. S. Campbell and A. K. Purdy, “Structure/function of human killer cell immunoglobulin-like receptors: Lessons from polymorphisms, evolution, crystal structures and mutations,” Immunology, vol. 132, no. 3. pp. 315-325, 2011, doi: 10.1111/j.1365-2567.2010.03398.x.

[42] D. Pende et al., “Killer Ig-like receptors (KIRs): Their role in NK cell modulation and developments leading to their clinical exploitation,” Frontiers in Immunology, vol. 10, no. May 2019, doi: 10.3389/fimmu.2019.01179.

[43] L. K. Ward-Kavanagh, W. W. Lin, J. R. Šedý, and C. F. Ware, “The TNF Receptor Superfamily in Co-stimulating and Co-inhibitory Responses,” Immunity, vol. 44, no. 5. pp. 1005-1019, 2016, doi: 10.1016/j.immuni.2016.04.019.

[44] C. M. Gonçalves, S. N. Henriques, R. F. Santos, and A. M. Carmo, “CD6, a rheostat-type signalosome that tunes T cell activation,” Frontiers in Immunology, vol. 9. 2018, doi: 10.3389/fimmu.2018.02994.

[45] M. Steri et al., “Overexpression of the cytokine BAFF and autoimmunity risk,” N. Engl. J. Med., vol. 376, no. 17, pp. 1615-1626, 2017, doi: 10.1056/NEJMoa1610528.

[46] M. Chen et al., “The function of BAFF on T helper cells in autoimmunity,” Cytokine and Growth Factor Reviews, vol. 25, no. 3. pp. 301-305, 2014, doi: 10.1016/j.cytogfr.2013.12.011.

[47] M. M. Varin, L. Le Pottier, P. Youinou, D. Saulep, F. Mackay, and J. O. Pers, “B-cell tolerance breakdown in Sjögren's Syndrome: Focus on BAFF,” Autoimmunity Reviews, vol. 9, no. 9. pp. 604-608, 2010, doi: 10.1016/j.autrev.2010.05.006.

[48] D. C. Wu, J. Yao, K. S. Ho, A. M. Lambowitz, and C. O. Wilke, “Limitations of alignment-free tools in total RNA-seq quantification,” BMC Genomics, vol. 19, no. 1, 2018, doi: 10.1186/s12864-018-4869-5.

[49] S. M. E. Sahraeian et al., “Gaining comprehensive biological insight into the transcriptome by performing a broad-spectrum RNA-seq analysis,” Nat. Commun., vol. 8, no. 1, 2017, doi: 10.1038/s41467-017-00050-4.

[50] C. Everaert et al., “Benchmarking of RNA-sequencing analysis workflows using whole-transcriptome RT-qPCR expression data,” Sci. Rep., vol. 7, no. 1, 2017, doi: 10.1038/s41598-017-01617-3.

[51] M. Teng et al., “A benchmark for RNA-seq quantification pipelines,” Genome Biol., vol. 17, no. 1, 2016, doi: 10.1186/s13059-016-0940-1.

[52] C. Robert and M. Watson, “Errors in RNA-Seq quantification affect genes of relevance to human disease,” Genome Biol., vol. 16, no. 1, 2015, doi: 10.1186/s13059-015-0734-x.

[53] M. J. Goldman et al., “Visualizing and interpreting cancer genomics data via the Xena platform,” Nature Biotechnology, vol. 38, no. 6. pp. 675-678, 2020, doi: 10.1038/s41587-020-0546-8.

[54] N. L. Bray, H. Pimentel, P. Melsted, and L. Pachter, “Near-optimal probabilistic RNA-seq quantification,” Nat. Biotechnol., vol. 34, no. 5, pp. 525-527, 2016, doi: 10.1038/nbt.3519.

[55] J. Harrow et al., “GENCODE: The reference human genome annotation for the ENCODE project,” Genome Res., vol. 22, no. 9, pp. 1760-1774, September 2012, doi: 10.1101/gr.135350.111.

[56] C. Soneson, M. I. Love, and M. D. Robinson, “Differential analyses for RNA-seq: transcript-level estimates improve gene-level inferences,” F1000Research, vol. 4, p. 1521, December 2015, doi: 10.12688/f1000research.7563.1.

[57] M. D. Robinson, D. J. McCarthy, and G. K. Smyth, “edgeR: A Bioconductor package for differential expression analysis of digital gene expression data,” Bioinformatics, vol. 26, no. 1, pp. 139-140, November 2009, doi: 10.1093/bioinformatics/btp616.

[58] M. D. Robinson and A. Oshlack, “A scaling normalization method for differential expression analysis of RNA-seq data,” Genome Biol., vol. 11, no. 3, p. R25, March 2010, doi: 10.1186/gb-2010-11-3-r25.

[59] A. Bird, “DNA methylation patterns and epigenetic memory,” Genes and Development, vol. 16, no. 1. pp. 6-21, 2002, doi: 10.1101/gad.947102.

[60] M. Melanie, “An introduction to genetic algorithms By Melanie Mitchell. MIT Press, Cambridge, Mass. (1996). 205 pages. $30.00,” Comput. Math. with Appl., vol. 32, no. 6, p. 133, 1996, doi: 10.1016/S0898-1221(96)90227-8.

[61] E. Willighagen, M. Ballings, and M. M. Ballings, “Package ‘genalg,’” 2015.

What is claimed is:

1. A method of identifying cell-type-specific gene expression levels for specific cells in a plurality of tissue samples, the method comprising:
(a) receiving a collection of bulk gene expression level measurements and cell fractions for each cell type in each of the tissue samples in a given collection of samples, obtained from a set of tissue samples of a plurality of cell types;
(b) performing high resolution deconvolution on the bulk gene expression measurements and cell fractions for each sample to generate a first output comprising predicted cell-type-specific gene expression levels in each cell type;
(c) ranking the first output with a confidence ranking system to generate a first output ranking of genes with predicted cell-type-specific gene expression levels of low or high confidence;
(d) performing hierarchical deconvolution the genes of low confidence from step (c) to generate a second output, such that for each gene of low confidence from step (c), the expression of the gene in a specific cell type is re-estimated by removing expression of genes of high confidence in all other cell types from the bulk gene expression measurements, thereby generating a second output comprising genes with predicted cell-type-specific gene expression levels of low or high confidence;
(e) ranking the second output with the confidence ranking system to generate a second output ranking of genes with predicted cell-type-specific gene expression levels of low or high confidence;
(f) performing imputation based deconvolution on the genes of low confidence from step (e) to generate a third output, such that for each gene of low confidence from step (e) for which gene expression levels in a particular cell type are highly correlated with the bulk gene expression levels of more than two genes of high confidence, a Lasso regression-based prediction model based on the bulk gene expression levels is applied, thereby imputing the expression of the low confidence genes based on the expression of high confidence genes from the first output ranking;
(g) ranking the third output with the unsupervised ranking system to generate a third output ranking comprising genes with predicted cell-type-specific gene expression levels of low or high confidence; and,
(h) generating a final confidence score for each pair of predicted gene expression levels and specific cell type, based on the predicted cell-type-specific gene expression levels of each gene of low or high confidence as determined in step
(g) in comparison to the predicted cell-type-specific gene expression level of each gene of high confidence as determined in step (g);
thereby generating deconvolved cell-type-specific gene expression data identifying cell-type-specific gene expression levels for each cell type in each of the individual tissue samples.

2. The method of claim 1, wherein the first output ranking, the second output ranking, and/or the third output ranking for each gene is a 1 or 0.

3. The method of claim 2, wherein a ranking of 1 indicates the output is high confidence and a ranking of 0 indicates the output is low confidence.

4. The method of claim 1, wherein the high resolution deconvolution comprises determining the cell types in which the genes are weakly expressed; recursively splitting the samples into finite sub-groups using p-freedom based splitting; and, performing ensemble sliding window deconvolution.

5. The method of claim 1, wherein the plurality of tissue samples is obtained from a set of tumor samples.

6. The method of claim 1, wherein the final confidence score for each gene-specific cell type is determined by: (i) for each gene, averaging the pair-wise correlations between the predicted gene expression levels of the gene in each specific cell type and the predicted expression level of genes of high confidence as determined in step (g) of claim 1; (ii) randomly shuffling the predicted cell-type-specific gene expression levels across the samples to generate a background and repeating step (i) to estimate a background distribution of scores for each gene; (iii) for each gene and each cell type, determining an empirical p-value pv based on the background distribution of scores; and, (iv) for each gene-cell type pair, subtracting pv from 1 to generate the final confidence score for the predicted gene expression levels of each gene in each specific cell type.

7. The method of claim 1, wherein the confidence ranking system comprises ranking a prediction based on each informative feature/measurement collected during or after each deconvolution step independently.

8. The method of claim 1, wherein the confidence ranking system uses the correlation between cell fraction and bulk expression, variations among groups from recursive splitting deconvolution, consistency between sliding windows and recursive splitting deconvolution for confidence ranking of the first output.

9. The method of claim 1, wherein the confidence ranking system uses the correlation between cell fraction and bulk expression, variations among groups from recursive splitting deconvolution, consistency between sliding windows and recursive splitting deconvolution for confidence ranking of the second output for the cell component that will be removed from bulk and mean correlations with highly predictable genes from the first output.

10. The method of claim 1 any of the preceding claims, wherein the confidence ranking system uses mean correlations with genes of high confidence from both the first output and the second output.

11. The method of claim 1 any of the preceding claims, wherein the cell fractions of each cell type are determined by receiving bulk gene expression measurements and cell-type-signature profiles; and, performing support vector machine (SVM) regression on the bulk gene expression measurements and cell-type-signature profiles; thereby determining the cell fractions of each cell type.

12. The method of claim 12, wherein determining the cell fractions of each cell type comprises performing batch correction on the bulk gene expression measurements and cell-type-signature profiles.

13. The method of claim 1, wherein the cell types comprise tumor, immune, and stromal cell types.

14. A method of identifying ligand-receptor interactions between a first cell type and a second cell type from a tissue sample, comprising:
(a) querying ligand-receptor interactions between the first cell type and second cell type from a database comprising a catalog of ligand-receptor interactions among a plurality of cell types and an expected distribution of ligand and receptors on each of the plurality of cell types, thereby generating a first list of potential ligand-receptor interactions between the first cell type and the second cell type;
(b) recursively adding to the first list ligands and receptors that are expected to be found on generic cell types related to the first and second cell types by function or lineage, or a combination thereof, thereby generating a second list of potential ligand-receptor interactions between the first cell type and the second cell type;
(c) receiving deconvolved cell-type-specific gene expression data according to any of the preceding claims;
(d) determining the likelihood of each potential ligand-receptor interaction between the first cell type and second cell type by assigning a binary score to the interaction, wherein based on the deconvolved cell-type-specific gene expression data, the binary score is 1 if the ligand is overexpressed in the first cell type and the receptor is overexpressed in the second cell type, and the binary score is 0 otherwise; and,
(e) inferring the activity of queried ligand-receptor interactions using the cell-type-specific gene expression levels,
thereby identifying ligand-receptor interactions between cell types.

15. The method of claim 14, wherein the tissue sample is a tumor sample.

16. The method of claim 14, wherein the ligand-receptor interactions comprise at least one of cytokine/chemokine - cytokine/chemokine receptor interactions, ligand-receptor interactions involved in cell adhesion/leukocyte trans-endothelial migration, ligand-receptor interactions involving the TNF receptor superfamily, and ligand receptor interactions involved in regulation of NK and T cell cytotoxicity.

17. The method of claim 14, wherein the ligand is overexpressed in the first cell type and the receptor is overexpressed in the second type in comparison to, based on the deconvolved cell-type gene expression data, the median deconvolved expression of the ligand in the first cell type and the median deconvolved expression of the receptor in the second cell type.

18. The method of claim 14, further comprising determining if a ligand-receptor interaction is more likely to occur in a tissue sample with a specific phenotype as compared to a control group, by computing an enrichment score, wherein the enrichment score is expressed as an odds ratio of the interaction in the specific phenotype, wherein a score around 1 indicates a neutral trend, a score>1 indicates enrichment of the interaction in the specific phenotype, and a score close to 0 indicates enrichment in the control group.

19. At least one non-transitory computer readable medium storing instructions which when executed by at least one processor, cause the at least one processor to:
(a) receive a collection of bulk gene expression level measurements and cell fractions for each cell type in a set of tissue samples of a plurality of cell types;
(b) perform high resolution deconvolution on the bulk gene expression measurements and cell fractions for each sample to generate a first output comprising predicted cell-type-specific gene expression levels in each cell type;
(c) rank the first output with a confidence ranking system to generate a first output ranking of genes with predicted cell-type-specific gene expression levels of low or high confidence;
(d) perform hierarchical deconvolution the genes of low confidence from step (c) to generate a second output, such that for each gene of low confidence from step (c), the expression of the gene in a specific cell type is re-estimated by removing expression of genes of high confidence in all other cell types from the bulk gene expression measurements, thereby generating a second output comprising genes with predicted cell-type-specific gene expression levels of low or high confidence;
(e) rank the second output with the confidence ranking system to generate a second output ranking of genes with predicted cell-type-specific gene expression levels of low or high confidence;
(f) perform imputation based deconvolution on the genes of low confidence from step (e) to generate a third output, such that for each gene of low confidence from step (e) for which gene expression levels in a particular cell type are highly correlated with the bulk gene expression levels of more than two genes of high confidence, a Lasso regression-based prediction model based on the bulk gene expression levels is applied, thereby imputing the expression of the low confidence genes based on the expression of high confidence genes from the first output ranking;
(g) rank the third output with the unsupervised ranking system to generate a third output ranking comprising genes with predicted cell-type-specific gene expression levels of low or high confidence; and,
(h) generate a final confidence score for each pair of predicted gene expression levels and specific cell type, based on the predicted cell-type-specific gene expression levels of each gene of low or high confidence as determined in step (g) in comparison to the predicted cell-type-specific gene expression level of each gene of high confidence as determined in step (g),
thereby generating deconvolved cell-type-specific gene expression data identifying cell-type-specific gene expression levels for each cell type in the tissue samples.

20. The at least one non-transitory computer readable medium of claim 18, storing further instructions which when executed by at least one processor, cause the at least one processor to perform one or more of the steps of any one of claims 2-18.
